SEQUENCE LISTING (15 sequences)

SEQ ID NO: 1 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 2 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 3 - protein, 76 aa, Homo sapiens
MDPQTAPSRALLLLLFLHLAFLGGRSHPLGSPGSASDLETSGLQEQRNHLQGKLSELQVEQTSLEPLQESPRPTGV

SEQ ID NO: 4 - protein, 32 aa, Homo sapiens
SPKMVQGSGCFGRKMDRISSSSGLGCKVLRRH

SEQ ID NO: 5 - protein, 153 aa, Homo sapiens [sequence omitted]

SEQ ID NO: 6 - protein, 126 aa, Homo sapiens
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLR

SEQ ID NO: 7 - protein, 98 aa, Homo sapiens
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALG

SEQ ID NO: 8 - protein, 28 aa, Homo sapiens
SLRRSSCFGGRMDRIGRQSGLGCNSFRY

SEQ ID NO: 9 - protein, 126 aa, Homosapiens
MHLSQLLACALLLTLLSLRPSEAKPGAPPKVPRTPPAEELAEPQAAGGGQKKGDKAPGGGGANLKGDRSRLLRDLRVDTKSRAAWARLLQEHPNARKYKGANKKGLSKGCFGLKLDRIGSMSGLGC

SEQ ID NO: 10 - protein, 103 aa, Homo sapiens
KPGAPPKVPRTPPAEELAEPQAAGGGQKKGDKAPGGGGANLKGDRSRLLRDLRVDTKSRAAWARLLQEHPNARKYKGANKKGLSKGCFGLKLDRIGSMSGLGC

SEQ ID NO: 11 - protein, 53 aa, Homo sapiens
DLRVDTKSRAAWARLLQEHPNARKYKGANKKGLSKGCFGLKLDRIGSMSGLGC

SEQ ID NO: 12 - protein, 29 aa, Homo sapiens
YKGANKKGLSKGCFGLKLDRIGSMSGLGC

SEQ ID NO: 13 - protein, 22 aa, Homo sapiens
GLSKGCFGLKLDRIGSMSGLGC

SEQ ID NO: 14 - protein, 38 aa, Homo sapiens
EVKYDPCFGHKIDRINHVSNLGCPSLRDPRPNAPSTSA

SEQ ID NO: 15 - protein, 50 aa, Homo sapiens
KPGAPPKVPRTPPAEELAEPQAAGGGQKKGDKAPGGGGANLKGDRSRLLR

(19) United States
(12) Patent Application Publication
(10) Pub. No.: US 2009/0286256 A1
(43) Pub. Date: Nov. 19, 2009

(54) ISOLATED HUMAN AUTOANTIBODIES TO NATRIURETIC PEPTIDES AND METHODS AND KITS FOR DETECTING HUMAN AUTOANTIBODIES TO NATRIURETIC PEPTIDES

(75) Inventors: Maciej Adamczyk, Gurnee, IL (US); Roy Jeffrey Brashear, Mundelein, IL (US); Phillip G. Mattingly, Third Lake, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 12/177,584

(22) Filed: Jul. 22, 2008

Related U.S. Application Data
(60) Provisional application No. 61/054,348, filed on May 19, 2008.

(51) Int. Cl. G01N 33/53; G01N 33/566; C07K 16/18

(52) U.S. Cl. 435/7.1; 436/501; 530/387.1

(57) ABSTRACT

The present disclosure relates to isolated human autoantibodies and assays and kits for detecting human autoantibodies reactive with at least one natriuretic peptide or natriuretic peptide fragment in a test sample.

### RELATED APPLICATION INFORMATION

This application claims the priority of Provisional U.S. Patent Application Ser. No. 61/054,348 filed on May 19, 2008, incorporated by reference in its entirety.

### TECHNICAL FIELD

The present disclosure relates to isolated human autoantibodies and assays and kits for detecting one or more human autoantibodies reactive with at least one natriuretic peptide or natriuretic peptide fragment in a test sample.

### BACKGROUND

A-type natriuretic peptide (hereinafter “ANP”), B-type natriuretic peptide (hereinafter “BNP”), C-type natriuretic peptide (hereinafter “CNP”) and Dendroaspis natriuretic peptide (hereinafter “DNP”) are each members of a family of hormones known as “natriuretic peptides”. ANP and BNP share a wide spectrum of biological properties and belong to the cardiac natriuretic system. Both ANP and BNP are of myocardial cell origin while CNP is of endothelial cell origin. DNP was isolated from the venom of the green mamba snake and possesses structural similarity to ANP, BNP and CNP.

ANP received its name because it is secreted by the heart in the atria. Initially, “ANP” stood for “atrial natriuretic peptide”. However, since ANP was found to belong to the cardiac natriuretic system, the word “atrial” was changed to “A-type”. The human version of ANP contains 151 amino acids and has a signal peptide sequence at its amino-terminal end. The pro-peptide is stored as a 126-amino acid peptide, proANP₁₋₁₂₆, which is produced by cleavage of the signal peptide. When appropriate signals for hormone release are given, proANP₁₋₁₂₆ is further split into an NH₂-terminal fragment, proANP₁₋₉₈, and the COOH-terminal peptide ANP₉₉₋₁₂₆ which is generally considered to the biologically active molecule.

BNP received its name because it was first isolated from porcine brain, thus, initially, “BNP” stood for “brain natriuretic peptide”. However, because BNP was found to belong to the cardiac natriuretic system, the word “brain” was changed to “B-type”. Therefore, “BNP” now refers to “B-type natriuretic peptide”. In humans, BNP is secreted by the heart through the coronary sinus, predominantly from the cardiac ventricles. The pre-pro peptide precursor of human BNP (hereinafter “human pre-proBNP”) is 134 amino acids in length (SEQ ID NO:1) and comprises a short signal peptide, which is enzymatically cleaved off to release the human pro peptide of BNP (hereinafter “human proBNP”) which is 108 amino acids in length (SEQ ID NO:2). Human proBNP is further cleaved into an N-terminal pro peptide of human BNP (hereinafter “human NT-proBNP”) which is 76 amino acids in length (SEQ ID NO:3) and the active hormone, human BNP (hereinafter “hBNP” or “hBNP-32”), which is 32 amino acids in length (SEQ ID NO:4). It has been suggested that each of human NT pro-BNP, hBNP-32, and human proBNP-can circulate in human plasma (See, Tateyama et al., Biochem. Biophys. Res. Commun., 185: 760-7 (1992); Hunt et al., Biochem. Biophys. Res. Commun. 214: 1175-83 (1995)).

Human CNP is a 126 amino acid peptide found in the brain and cerebral spinal fluid and is the most prevalent of the three peptides in the central nervous system. Little if any CNP is present in the heart.

As mentioned previously, DNP was isolated from the venom of the green mamba snake. The mature form of DNP is made up of 38 amino acids. DNP-like immunoreactivity (DNP-LI) has been reported in human plasma and the plasma concentration of DNP-LI has been found to be elevated in patients with congestive heart failure (See, Cataliotti, et al., Mayo Clin. Proc., 76:111-1119 (2001)). Additionally, it is also known that the infusion of synthetic DNP results in marked natriuresis and diuresis in association with increased plasma and urinary cyclic guanosine monophosphate. Id.

In humans, heart disease can stimulate the secretion of ANP and BNP. In fact, the secretion of ANP and BNP in humans typically reflects a change in cardiac function. Specifically, the secretion of ANP is typically accelerated when the atrium undergoes a load, while the biosynthesis and secretion of BNP is stimulated when the ventricle undergoes a load. Thereupon, both ANP and BNP are useful as indicators in the diagnosis of heart disease. However, despite this and over time, BNP has become recognized as a useful indicator in the diagnosis of heart disease, more so than ANP. For example, the blood concentration of BNP is only ⅙ of ANP in a so-called normal subject but it becomes higher than ANP in patients of heart failure. Moreover, the blood concentration of BNP increases in the case of heart failure like ANP, and the plasma concentration of BNP often exceeds that of ANP, thus reflecting more accurately the severity of heart dysfunction. Moreover, BNP level in patients experiencing heart failure sometimes increases to several tens times to several hundreds times of that of healthy subjects. The clinical utility of BNP as a diagnostic marker has been reported in numerous documents (see, e.g., U.S. Pat. Nos. 5,786,163, 6,117,644, 6,162,902, 6,376,207, 6,677,124, and 6,461,828 reissued as RE39,816; WO 2002/089657; European Patent Nos. EP0542255 and EP01016867).

Assays, in particular, immunoassays, employ one or more antibodies that react with an analyte of interest to form an immunocomplex in a quantity dependent on the concentration of the analyte of interest. The antibodies used in immunoassays for analytes of interest in human test samples are typically derived from another species, such as, for example, mouse, goat, rabbit, etc. In some instances, the human test subject also produces antibodies that react with the analyte of interest to form an immunocomplex. Such human-immunocomplexes may confound the results of assays for an analyte of interest, providing inaccurate quantitation.

Additionally, if the human test subject produces antibodies that react with the analyte of interest, and the analyte of interest is an endogenous substance, the test subject may be predisposed to certain clinical manifestations relating to an autoimmune disease. Such endogenous human antibodies reactive with endogenous human antigens are referred to as autoantibodies. Using methods well-known in the art, the amino acid sequence of such autoantibodies can be determined along with the corresponding nucleic acid sequence of the genes controlling their production. Such information is useful, for instance, in the design of pharmaceutical compositions (e.g., since recombinant and chimeric antibodies can greatly facilitate screening). Therefore, given the importance of natriuretic peptide levels in association with certain diseases, such as, but not limited to heart disease, a need remains for new natriuretic peptide assays and kits that accurately quantitate the levels of human autoantibodies to such peptides in a subject. These and other objects and advantages of the disclosure will be apparent from the further description provided herein.

### SUMMARY

In one embodiment, the present disclosure relates to a method for detecting one or more autoantibodies reactive with at least one natriuretic peptide or natriuretic peptide fragment in a test sample. The method comprises the steps of:

(a) preparing a mixture comprising a test sample being assessed for the presence of one or more autoantibodies to at least one natriuretic peptide or natriuretic peptide fragment, and a first specific binding partner labeled with a detectable label wherein the first specific binding partner is a natriuretic peptide or a natriuretic peptide fragment and further wherein the one or more autoantibodies and the first specific binding partner form a first specific binding partner-autoantibody complex; and

(b) measuring the signal generated by or emitted from the detectable label. Optionally, this method (and all other methods described herein) are adapted for use in an automated system or semi-automated system.

In the above method, the natriuretic peptide is a pre-pro peptide precursor of human ANP, a pro peptide of human ANP, a N-terminal pro peptide of ANP, human ANP, a pre-pro peptide precursor of human BNP, a pro peptide of human BNP, a N-terminal pro peptide of BNP, human BNP, human CNP, a pro peptide of human CNP, Dendroaspis natriuretic peptide, a natriuretic peptide fragment or any combinations thereof.

In the above method, the preferred detectable label is an acridinium compound. In one aspect, the acridinium compound is an acridinium-9-carboxamide having a structure according to formula I:

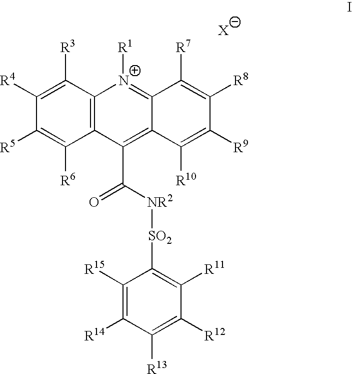

wherein R¹ and R² are each independently selected from the group consisting of: alkyl, alkenyl, alkynyl, aryl or aralkyl, sulfoalkyl, carboxyalkyl and oxoalkyl; and

wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and

optionally, if present,

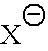

is an anion.

In another aspect, the acridinium compound is an acridinium-9-carboxylate aryl ester having a structure according to formula II:

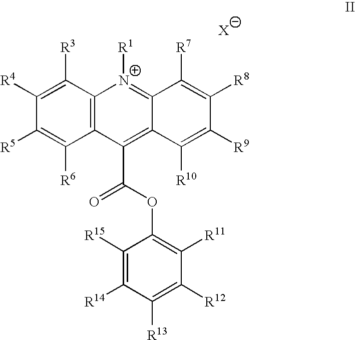

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present,

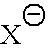

is an anion.

In the above method, the method can further comprise the steps of:

generating in or providing to the mixture a source of hydrogen peroxide before or after the addition of the first specific binding partner containing the detectable label in step (a);

adding a basic solution to the mixture to generate a light signal; and

measuring the signal in step (b) by measuring the light generated to detect the one or more autoantibodies.

In another embodiment, the present disclosure relates to a method for detecting one or more autoantibodies reactive with at least one natriuretic peptide or natriuretic peptide fragment in a test sample. The method comprises the steps of:

(a) preparing a mixture comprising a test sample being assessed for the presence of one or more autoantibodies to at least one natriuretic peptide or natriuretic peptide fragment and a first specific binding partner that is immobilized on a solid phase, wherein the first specific binding partner is a natriuretic peptide or a natriuretic peptide fragment and further wherein the one or more autoantibodies and the first specific binding partner form a solid phase first specific binding partner-autoantibody complex;

(b) adding a second specific binding partner labeled with a detectable label to the mixture to form a first specific binding partner-one or more autoantibodies-second specific binding partner complex, wherein the second specific binding partner is an anti-human antibody; and

(c) measuring the signal generated by or emitted from the detectable label.

Optionally the method further comprises an additional step selected from the group consisting of: (1) removing any unbound one or more autoantibodies from the solid phase first specific binding partner-autoantibody complex prior to step (b); and (2) removing any unbound second specific binding partner labeled with a detectable label from the first specific binding partner-one or more autoantibodies-second specific binding partner complex prior to step (c). Moreover, the method further optionally comprises the steps of:

(i) generating in or providing to the mixture a source of hydrogen peroxide before or after the addition of the second specific binding partner containing the detectable label in step (b);

(ii) after step (b) and before step (c), adding a basic solution to the mixture to generate a light signal; and

(iii) measuring the signal in step (c) by measuring the light generated to detect the one or more autoantibodies.

Thus, the present disclosure also relates to a method for detecting one or more autoantibodies reactive with at least one natriuretic peptide or natriuretic peptide fragment in a test sample. The method comprises the steps of:

(a) preparing a mixture comprising a test sample being assessed for the presence of one or more autoantibodies to at least one natriuretic peptide or natriuretic peptide fragment and a first specific binding partner that is immobilized on a solid phase, wherein the first specific binding partner is a natriuretic peptide or a natriuretic peptide fragment and further wherein the one or more autoantibodies and the first specific binding partner form a solid phase first specific binding partner-one or more autoantibodies complex;

(b) removing any unbound one or more autoantibodies from the solid phase first specific binding partner-autoantibody complex;

(c) adding a second specific binding partner labeled with a detectable label to the mixture to form a first specific binding partner-one or more autoantibodies-second specific binding partner complex, wherein the second specific binding partner is an anti-human antibody;

(d) removing any unbound second specific binding partner labeled with a detectable label from the first specific binding partner-one or more autoantibodies-second specific binding partner complex; and

(e) measuring the signal generated by or emitted from the detectable label.

In one embodiment, the method relates the amount of signal in step (e) to the amount of the one or more autoantibodies in the test sample either by use of a standard curve for the analyte, or by comparison to a reference standard.

In the above method, the natriuretic peptide is a pre-pro peptide precursor of human ANP, a pro peptide of human ANP, a N-terminal pro peptide of ANP, human ANP, a pre-pro peptide precursor of human BNP, a pro peptide of human BNP, a N-terminal pro peptide of BNP, human BNP, human CNP, a pro peptide of human CNP, Dendroaspis natriuretic peptide, a natriuretic peptide fragment or any combinations thereof.

In the above method, the preferred detectable label is an acridinium compound. In one aspect, the acridinium compound is an acridinium-9-carboxamide having a structure according to formula I:

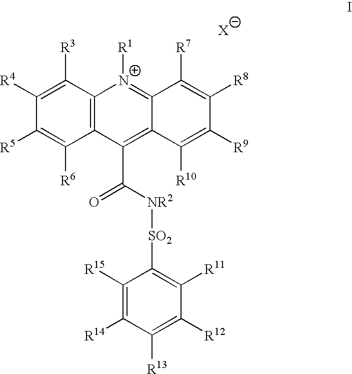

wherein R¹ and R² are each independently selected from the group consisting of: alkyl, alkenyl, alkynyl, aryl or aralkyl, sulfoalkyl, carboxyalkyl and oxoalkyl; and

wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and

optionally, if present,

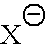

is an anion.

In another aspect, the acridinium compound is an acridinium-9-carboxylate aryl ester having a structure according to formula II:

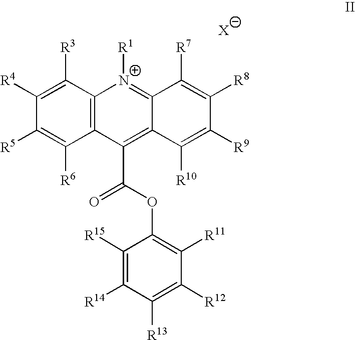

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present,

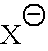

is an anion.

In the above method, the method can further comprise the steps of:

generating in or providing to the mixture a source of hydrogen peroxide before or after the addition of the second specific binding partner containing the detectable label after step (d);

adding a basic solution to the mixture to generate a light signal; and

measuring the signal in step (e) by measuring the light generated to detect the autoantibody.

In one embodiment, the method relates the amount of signal in step (e) to the amount of the one or more autoantibodies in the test sample either by use of a standard curve for the analyte, or by comparison to a reference standard.

In another embodiment, the present disclosure relates to a kit for detecting one or more autoantibodies reactive to at least one natriuretic peptide or natriuretic peptide fragment, in a test sample. The kit can comprise:

(a) at least one natriuretic peptide or natriuretic peptide fragment;

(b) at least one detectable label; and

(c) instructions for detecting said one or more autoantibodies.

The above kit can also further comprise at least one anti-human antibody. Also, in the above kit, the natriuretic peptide is a pre-pro peptide precursor of human ANP, a pro peptide of human ANP, N-terminal pro peptide of ANP, human ANP, a pre-pro peptide precursor of human BNP, a pro peptide of human BNP, N-terminal pro peptide of BNP, human BNP, human CNP, a pro peptide of human CNP, Dendroaspis natriuretic peptide, a natriuretic peptide fragment or any combinations thereof.

In the above kit, the detectable label can be an acridinium compound. In one aspect, the acridinium compound is an acridinium-9-carboxamide having a structure according to formula I:

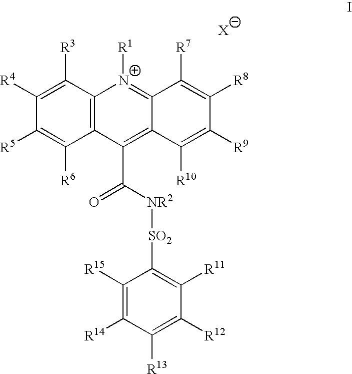

wherein R¹ and R² are each independently selected from the group consisting of: alkyl, alkenyl, alkynyl, aryl or aralkyl, sulfoalkyl, carboxyalkyl and oxoalkyl; and

wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, carboxyalkyl and oxoalkyl; and

optionally, if present,

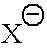

is an anion.

In another aspect, the acridinium compound is an acridinium-9-carboxylate aryl ester having a structure according to formula II:

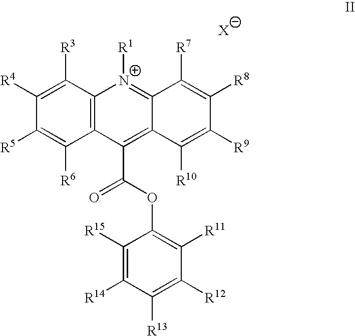

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and, optionally, if present,

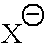

is an anion.

In addition, the kit can also contain additional components. For example, the kit can also further comprise:

(d) at least one basic solution; and

(e) a source of hydrogen peroxide, wherein said source contains a predetermined amount of hydrogen peroxide.

Moreover, in addition to the at least one basic solution and the source of hydrogen peroxide, the kit can also contain at least one anti-human antibody.

In still yet another embodiment, the present disclosure relates to an isolated human natriuretic peptide autoantibody that is obtained by a process comprising the steps of:

(a) preparing a mixture comprising a human natriuretic peptide autoantibody; and

(b) isolating the human natriuretic peptide autoantibody from the mixture.

The above identified antibody can be an IgG antibody. Additionally, the antibody can be an isolated human autoantibody selected from the group consisting of: a pre-pro peptide precursor of human BNP autoantibody, a pro peptide of human BNP autoantibody, a N-terminal pro peptide of BNP autoantibody and a human BNP autoantibody. Specifically, the antibody can be an isolated pro peptide human BNP autoantibody.

In still yet another embodiment, the present disclosure relates to a method of screening for at least one agent useful in inhibiting the binding of at least one human natriuretic peptide or natriuretic peptide fragment to at least one human natriuretic peptide autoantibody. The method comprises the steps of:

(a) preparing a mixture comprising an isolated human natriuretic peptide autoantibody;

(b) adding to the mixture at least one human natriuretic peptide or natriuretic peptide fragment and at least one agent to be tested; and

(c) determining whether the agent inhibits the binding of the at least one human natriuretic peptide or natriuretic peptide fragment to the human natriuretic peptide autoantibody.

In still yet another embodiment, the present disclosure relates to a method of determining the reliability of a human natriuretic peptide assay result. The method comprises the steps of:

(a) assaying a test sample for one or more autoantibodies reactive with a human natriuretic peptide; and

(b) determining the reliability of a human natriuretic peptide assay result, wherein the presence of an elevated level in the test sample of one or more autoantibodies reactive with a human natriuretic peptide indicates that the human natriuretic peptide result is not reliable.

In still yet a further embodiment, the present disclosure relates to a method of assessing whether a subject has or is at risk of developing cardiovascular disease. The method comprises the steps of:

(a) determining the concentration or amount in a test sample from a subject of one or more autoantibodies reactive with human natriuretic peptide; and

(b) comparing the concentration or amount of the one or more autoantibodies reactive with human natriuretic peptide determined in step (a) with a predetermined level, wherein if the concentration or amount of the one or more autoantibodies reactive with human natriuretic peptide determined in step (a) is favorable with respect to a predetermined level, then the subject is determined not to have or be at risk for a cardiovascular disease; and further wherein if the concentration or amount of the one or more autoantibodies reactive with human natriuretic peptide determined in step (a) is unfavorable with respect to the predetermined level then the subject is determined to have or be at risk for a cardiovascular disease.

In the above method, the human natriuretic peptide is a pre-pro peptide precursor of human ANP, a pro peptide of human ANP, a N-terminal pro peptide of ANP, human ANP, a pre-pro peptide precursor of human BNP, a pro peptide of human BNP, a N-terminal pro peptide of BNP, human BNP, human CNP, a pro peptide of human CNP, a natriuretic peptide fragment or any combinations thereof.

In still another embodiment, the present disclosure relates to a method of monitoring the progression of disease (e.g., cardiovascular disease) in a subject, the method comprising the steps of:

(a) determining the concentration or amount in a first test sample from a subject of one or more autoantibodies reactive with human natriuretic peptide;

(b) determining the concentration or amount in a second or subsequent test sample from the subject of one or more autoantibodies reactive with human natriuretic peptide; and

(c) comparing the concentration or amount of one or more autoantibodies reactive with human natriuretic peptide determined in step (b) with the concentration or amount of one or more autoantibodies reactive with human natriuretic peptide determined in step (a), wherein if the concentration or amount determined in step (b) is unchanged or is unfavorable when compared to the concentration or amount of one or more autoantibodies reactive with human natriuretic peptide determined in step (a), then the disease in the subject is determined to have continued, progressed or worsened, further wherein, if the concentration or amount of one or more autoantibodies reactive with human natriuretic peptide determined in step (b) is favorable when compared to the concentration or amount of one or more autoantibodies reactive with human natriuretic peptide determined in step (a), then the disease in the subject is determined to have discontinued, regressed or improved.

This method further optionally comprises comparing the concentration or amount of one or more autoantibodies reactive with human natriuretic peptide determined in step (b) or step (d) with a predetermined level. Additionally, the method optionally comprises treating the subject with one or more pharmaceutical compositions for a period of time if the comparison shows that the concentration or amount of one or more autoantibodies reactive with human natriuretic peptide determined in step (b) or step (d) is unfavorably altered with respect to the predetermined level.

In still yet another embodiment, the present disclosure relates to a method of monitoring treatment in a subject, e.g., treatment of a subject for cardiovascular disease by administration of a pharmaceutical composition. The method comprises the steps of:

(a) determining the concentration or amount in a first test sample from a subject of one or more autoantibodies reactive with human natriuretic peptide;

(b) treating the subject with one or more pharmaceutical compositions for a period of time;

(c) determining the concentration or amount in a second or subsequent test sample obtained from the subject following treatment in step (b) of one or more autoantibodies reactive with human natriuretic peptide; and

(d) comparing the concentration or amount of one or more autoantibodies reactive with human natriuretic peptide determined in step (c) with the concentration or amount of one or more autoantibodies reactive with human natriuretic peptide determined in step (a),

wherein if the concentration or amount determined in step (c) is unchanged or is unfavorable when compared to the concentration or amount of one or more autoantibodies reactive with human natriuretic peptide determined in step (a), then the disease in the subject is determined to have continued, progressed or worsened, and the subject should be treated with a higher concentration of the one or more pharmaceutical compositions administered to the subject in step (b) or the subject should be treated with one or more pharmaceutical compositions that are different then the one or more pharmaceutical compositions administered to the subject in step (b),

further wherein, if the concentration or amount of one or more autoantibodies reactive with human natriuretic peptide determined in step (c) is favorable when compared to the concentration or amount of one or more autoantibodies reactive with human natriuretic peptide determined in step (a), then the disease in the subject is determined to have discontinued, regressed or improved, and the subject should continue to be administered the one or pharmaceutical compositions of step (b).

Moreover, the present disclosure also relates to methods of determining whether a subject predisposed to or suffering from a disease (e.g., cardiovascular disease) will benefit from treatment with a drug, and the response of a subject receiving treatment by monitoring one or more autoantibodies reactive with human natriuretic peptide. In particular, the disclosure relates to natriuretic peptide companion diagnostic methods and products.

### BRIEF DESCRIPTION OF THE FIGURE

FIG. 1 shows a distribution plot indicating the range of anti-human proBNP autoantibody reactivity in three populations using the methodology described in Example 2. As shown in FIG. 1, Δ represents a population that tested positive for human BNP, □ represents population that tested positive for human cardiac troponin-I (cTnI) and ∘ represents population of normal blood donors (ND); closed symbols represent highly reactive samples within each population, i.e., RLUmax>upper quartile plus 1.5 times the interquartile range.

### DETAILED DESCRIPTION

The present disclosure is based on the surprising and unexpected discovery by the inventors of presence of certain endogenous antibodies (autoantibodies) in test samples. Specifically, the autoantibodies discovered by the inventors are antibodies that are reactive with natriuretic peptides, such as B-type natriuretic peptides, and especially human proBNP, in human serum and plasma. The identification of these autoantibodies is significant in that the presence of other types of autoantibodies in test samples has been observed to contribute to the generation of false negative results in certain studies, such as in cardiac biomarker studies in troponin assays (See, for example, Bohner et al., Clin. Chem., 42, 2046 (1996)). Thus, the present disclosure provides methods for detecting autoantibodies to natriuretic peptides and natriuretic peptide fragments. These methods can be used to determine the reliability of a natriuretic peptide or natriuretic peptide fragment assay and to correctly determine the amount of natriuretic peptides or natriuretic peptide fragments in a test sample.

Moreover, the methods of the present disclosure can be used independently of a natriuretic peptide or natriuretic peptide fragment assay apart from correctly determining the amount of natriuretic peptides or natriuretic peptide fragments in a test sample. More specifically, the methods of the present disclosure can be used for detecting the presence of autoantibodies to natriuretic peptides and natriuretic peptide fragments in a test sample obtained from a subject. The identification of autoantibodies in a test sample has clinical significance in the diagnosis and monitoring of autoimmune diseases, and in assessing risk of autoimmune disease, among others. Particularly advantageous, the testing methods described herein can be employed for testing of a subject that exhibits symptoms of disease (e.g., cardiovascular disease), as well as of a subject that is apparently healthy and does not yet exhibit symptoms of disease (e.g., cardiovascular disease), but may with time.

Finally, the present disclosure also relates to isolated human natriuretic peptide autoantibodies.

### A. Definitions

Section headings as used in this section and the entire disclosure herein are not intended to be limiting.

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

a) Acyl (and Other Chemical Structural Group Definitions)

As used herein, the term “acyl” refers to a —C(O)Rₐ group where Rₐ is hydrogen, alkyl, cycloalkyl, cycloalkylalkyl, phenyl or phenylalkyl. Representative examples of acyl include, but are not limited to, formyl, acetyl, cylcohexylcarbonyl, cyclohexylmethylcarbonyl, benzoyl, benzylcarbonyl and the like.

As used herein, the term “alkenyl” means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

As used herein, the term “alkyl” means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

As used herein, the term “alkyl radical” means any of a series of univalent groups of the general formula CₙH₂ₙ₊₁ derived from straight or branched chain hydrocarbons.

As used herein, the term “alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

As used herein, the term “alkynyl” means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

As used herein, the term “amido” refers to an amino group attached to the parent molecular moiety through a carbonyl group (wherein the term “carbonyl group” refers to a —C(O)— group).

As used herein, the term “amino” means —NR_{b}R_{c}, wherein R_{b} and R_{c} are independently selected from the group consisting of hydrogen, alkyl and alkylcarbonyl.

As used herein, the term “anion” refers to an anion of an inorganic or organic acid, such as, but not limited to, hydrochloric acid, hydrobromic acid, sulfuric acid, methane sulfonic acid, formic acid, acetic acid, oxalic acid, succinic acid, tartaric acid, mandelic acid, fumaric acid, lactic acid, citric acid, glutamic acid, aspartic acid, phosphate, trifluoromethansulfonic acid, trifluoroacetic acid and fluorosulfonic acid and any combinations thereof.

As used herein, the term “aralkyl” means an aryl group appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

As used herein, the term “aryl” means a phenyl group, or a bicyclic or tricyclic fused ring system wherein one or more of the fused rings is a phenyl group. Bicyclic fused ring systems are exemplified by a phenyl group fused to a cycloalkenyl group, a cycloalkyl group, or another phenyl group. Tricyclic fused ring systems are exemplified by a bicyclic fused ring system fused to a cycloalkenyl group, a cycloalkyl group, as defined herein or another phenyl group. Representative examples of aryl include, but are not limited to, anthracenyl, azulenyl, fluorenyl, indanyl, indenyl, naphthyl, phenyl, and tetrahydronaphthyl. The aryl groups of the present disclosure can be optionally substituted with one-, two, three, four, or five substituents independently selected from the group consisting of alkoxy, alkyl, carboxyl, halo, and hydroxyl.

As used herein, the term “carboxy” or “carboxyl” refers to —CO₂H or —CO₂⁻.

As used herein, the term “carboxyalkyl” refers to a —(CH₂)ₙCO₂H or —(CH₂)ₙCO₂⁻ group where n is from 1 to 10.

As used herein, the term “cyano” means a —CN group.

As used herein, the term “cycloalkenyl” refers to a non-aromatic cyclic or bicyclic ring system having from three to ten carbon atoms and one to three rings, wherein each five-membered ring has one double bond, each six-membered ring has one or two double bonds, each seven- and eight-membered ring has one to three double bonds, and each nine- to ten-membered ring has one to four double bonds. Representative examples of cycloalkenyl groups include cyclohexenyl, octahydronaphthalenyl, norbornylenyl, and the like. The cycloalkenyl groups can be optionally substituted with one, two, three, four, or five substituents independently selected from the group consisting of alkoxy, alkyl, carboxyl, halo, and hydroxyl.

As used herein, the term “cycloalkyl” refers to a saturated monocyclic, bicyclic, or tricyclic hydrocarbon ring system having three to twelve carbon atoms. Representative examples of cycloalkyl groups include cyclopropyl, cyclopentyl, bicyclo[3.1.1]heptyl, adamantyl, and the like. The cycloalkyl groups of the present disclosure can be optionally substituted with one, two, three, four, or five substituents independently selected from the group consisting of alkoxy, alkyl, carboxyl, halo, and hydroxyl.

As used herein, the term “cycloalkylalkyl” means a —R_{d}Rₑ group where R_{d} is an alkylene group and Rₑ is cycloalkyl group. A representative example of a cycloalkylalkyl group is cyclohexylmethyl and the like.

As used herein, the term “halogen” means a —Cl, —Br, —I or —F; the term “halide” means a binary compound, of which one part is a halogen atom and the other part is an element or radical that is less electronegative than the halogen, e.g., an alkyl radical.

As used herein, the term “hydroxyl” means an —OH group.

As used herein, the term “nitro” means a —NO₂ group.

As used herein, the term “oxoalkyl” refers to —(CH₂)ₙC(O)Rₐ, where Rₐ is hydrogen, alkyl, cycloalkyl, cycloalkylalkyl, phenyl or phenylalkyl and where n is from 1 to 10.

As used herein, the term “phenylalkyl” means an alkyl group which is substituted by a phenyl group.

As used herein, the term “sulfo” means a —SO₃H or —SO₃⁻ group.

As used herein, the term “sulfoalkyl” refers to a —(CH₂)ₙSO₃H or —(CH₂)ₙSO₃⁻ group where n is from 1 to 10.

b) Antibody

As used herein, the term “antibody” refers to an immunoglobulin molecule or immunologically active portion thereof, namely, an antigen-binding portion. Examples of immunologically active portions of immunoglobulin molecules include F(ab) and F(ab′)₂ fragments which can be generated by treating an antibody with an enzyme, such as pepsin. Examples of antibodies that can be used in the present disclosure include, but are not limited to, polyclonal antibodies, monoclonal antibodies, chimeric antibodies, human antibodies, humanized antibodies, recombinant antibodies, single-chain Fvs (“scFv”), an affinity maturated antibody, single chain antibodies, single domain antibodies, F(ab) fragments, F(ab′) fragments, disulfide-linked Fvs (“sdFv”), and antiidiotypic (“anti-Id”) antibodies and functionally active epitope-binding fragments of any of the above. For simplicity sake, an antibody against an analyte is frequently referred to as being either an “anti-analyte antibody”, or merely an “analyte antibody” (e.g., a human natriuretic peptide antibody or a human natriuretic peptide autoantibody).

c) Autoantibody

As used herein, the terms “autoantibody” or “autoantibodies” refers an endogenous antibody or antibodies that binds to (or is “reactive with”) analyte that is naturally occurring in the subject in which the antibody is produced. Preferably, the analyte is a natriuretic peptide or a natriuretic peptide fragment.

d) Cardiovascular Disease (and Other Disease-Related Definitions)

As used herein, the phrase “cardiovascular disease” refers to various clinical diseases, disorders or conditions involving the heart, blood vessels or circulation. The diseases, disorders or conditions may be due to atherosclerotic impairment of coronary, cerebral or peripheral arteries. Cardiovascular disease includes, but is not limited to, coronary artery disease, peripheral vascular disease, hypertension, myocardial infarction, heart failure, etc. For example, in heart failure, “increased severity” of cardiovascular disease refers to the worsening of disease as indicated by increased NYHA classification, to, for example, Class III or Class IV and “reduced severity” of cardiovascular disease refers to an improvement of the disease as indicated by reduced NYHA classification, from, for example, class III or IV to class II or I.

As used herein, the phrase “heart failure” refers to a condition in which the heart cannot pump blood efficiently to the rest of the body. Heart failure may be due to damage to the heart or narrowing of the arteries due to infarction, cardiomyopathy (primary or secondary), hypertension, coronary artery disease, valve disease, birth defects or infection. Heart failure can further be described as chronic, congestive, acute, decompensated, systolic or diastolic. The New York Heart Association (NYHA) classification describes the severity of the disease based on functional capacity of the patient; NYHA class can progress and/or regress based on treatment or lack of response to treatment.

Cardiovascular disease as used herein includes cardiovascular autoimmune disease. “Cardiovascular autoimmune disease” as used herein refers to any deviation from a healthy or normal condition of the heart that is due to an underlying autoimmune disease, including any structural or functional abnormality of the heart, or of the blood vessels supplying the heart, that impairs its typical functioning. Examples of cardiovascular autoimmune diseases include myocarditis, cardiomyopathy, and ischemic heart disease, each due to an underlying autoimmune disease.

“Autoimmune disease” refers to the loss of immunological tolerance to self antigens. Some criteria for a diagnosis of autoimmune disease include: (1) the presence of circulating autoantibodies; (2) autoantibodies observed in the affected organ; (3) target antigen identified; (4) inducible in an animal model either by immunization with antigen, serum, or autoantibody transfer; and (5) responsive to immunosuppressive therapy or immunoabsorption. Other characteristics of autoimmune disease include its: (a) increased prevalence in women; (b) familial clustering (although this varies with disease); (c) asymptomatic risk (i.e., the presence of autoantibodies may precede the disease by years); (d) periodic nature; and (e) chronic nature.

“Autoimmunity” refers to one or more immune responses directed against host antigens, characterized, for example, by the presence of autoantibodies or T lymphocytes reactive with host antigens.

The term “myocarditis” refers to inflammation of the myocardium. Myocarditis can be caused by a variety of conditions such as viral infection, sarcoidosis, rheumatic fever, autoimmune diseases (such as systemic lupus erythematosus, etc.), and pregnancy.

The term “cardiomyopathy” refers to a weakening of the heart muscle or a change in heart muscle structure. It is often associated with inadequate heart pumping or other heart function abnormalities. Cardiomyopathy can be caused by viral infections, heart attacks, alcoholism, long-term, severe high blood pressure, nutritional deficiencies (particularly selenium, thiamine, and L-carnitine), systemic lupus erythematosus, celiac disease, and end-stage kidney disease. Types of cardiomyopathy include dilated cardiomyopathy, hypertrophic cardiomyopathy, and restrictive cardiomyopathy.

As used herein, the term “dilated cardiomyopathy” refers to a global, usually idiopathic, myocardial disorder characterized by a marked enlargement and inadequate function of the left ventricle. Dilated cardiomyopathy includes ischemic cardiomyopathy, idiopathic cardiomyopathy, hypertensive cardiomyopathy, infectious cardiomyopathy, alcoholic cardiomyopathy, toxic cardiomyopathy, and peripartum cardiomyopathy.

As used herein, the term “hypertrophic cardiomyopathy” refers to a condition resulting from the right and left heart muscles growing to be different sizes.

As used herein, the term “restrictive cardiomyopathy” refers to a condition characterized by the heart muscle's inability to relax between contractions, which prevents it from filling sufficiently.

The term “ischemic heart disease” refers to any condition in which heart muscle is damaged or works inefficiently because of an absence or relative deficiency of its blood supply; most often caused by atherosclerosis, it includes angina pectoris, acute myocardial infarction, and chronic ischemic heart disease.

“Angina pectoris” refers to chest discomfort caused by inadequate blood flow through the blood vessels (coronary vessels) of the myocardium.

A “myocardial infarction” (heart attack) occurs when an area of heart muscle dies or is damaged because of an inadequate supply of oxygen to that area.

e) Clinical Indicia

As used herein, the phrase “clinical indicia” refers to assays, test methods (such as imaging), standards (such as The New York Heart Association (NYHA) classification), biophysical measures (such as LDL concentration, HDL concentration, triglyceride concentration, blood pressure, body mass index, waist circumference, heart rate, fasting insulin concentration, fasting glucose concentration, diabetes status) and other biometric parameters (such as, but not limited to, race, gender, age, tobacco smoking status, previous history of cardiovascular disease, family history of cardiovascular disease, use of high blood pressure medication etc.) that provide an indicator of cardiovascular disease.

f) Dendroaspis Natriuretic Peptide (DNP) and Fragment

As used herein, the terms “Dendroaspis natriuretic peptide”, “DNP”, “DNP peptide”, or “DNP polypeptide” as used interchangeably herein, refer to a 38 amino acid molecule having the amino acid sequence shown in SEQ ID NO:14.

As used herein, the terms “Dendroaspis natriuretic peptide fragment”, “DNP fragment” or “DNP peptide fragment” as used interchangeably herein, refer to a polypeptide that comprises a fragment of a DNP peptide that contains a contiguous or nonlinear epitope of the DNP peptide. The precise boundaries of such an epitope fragment can be confirmed using ordinary skill in the art. In one aspect, a DNP fragment or DNP peptide fragment refers to a peptide that comprises at least five contiguous amino acids of SEQ ID NO:14. In another aspect, a DNP fragment or DNP peptide fragment refers to a peptide that comprises at least ten contiguous amino acids residues of SEQ ID NO:14; at least fifteen contiguous amino acids residues of amino acids of SEQ ID NO:14; at least 20 contiguous amino acids residues of SEQ ID NO:14; at least 25 contiguous amino acids residues of SEQ ID NO:14 or at least 30 contiguous amino acid residues of amino acids of SEQ ID NO:14.

g) Human A-Type Natriuretic Peptide (hANP) and Fragment

As used herein, the phrases “human A-Type natriuretic peptide”, “human ANP”, “hANP”, “hANP peptide”, or “hANP polypeptide” as used interchangeably herein, refer to a 28 amino acid molecule having the amino acid sequence shown in SEQ ID NO:8.

As used herein, the phrases “human A-Type natriuretic peptide fragment”, “hANP fragment” or “hANP peptide fragment” as used interchangeably herein, refer to a polypeptide that comprises a fragment of an hANP peptide that contains a contiguous or nonlinear epitope of the hANP peptide. The precise boundaries of such an epitope fragment can be confirmed using ordinary skill in the art. In one aspect, an hANP fragment or hANP peptide fragment refers to a peptide that comprises at least five contiguous amino acids of SEQ ID NO:8. In another aspect, a hANP fragment or hANP peptide fragment refers to a peptide that comprises at least ten contiguous amino acids residues of SEQ ID NO:8; at least fifteen contiguous amino acids residues of amino acids of SEQ ID NO:8 or at least 20 contiguous amino acids residues of SEQ ID NO:8. Examples of hANP fragments or hANP peptide fragments include, but are not limited to, amino acid sequences containing amino acids residues 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 1-9, 1-8, 1-7, 1-6, 1-5, 2-28, 2-27, 2-26, 2-25, 2-24, 2-23, 2-22, 2-21, 2-20, 2-19, 2-18, 2-17, 2-16, 2-15, 2-14, 2-13, 2-12, 2-11, 2-10, 2-9, 2-8, 2-7, 2-6, 3-28, 3-27, 3-26, 3-25, 3-3-24, 3-23, 3-22, 3-21, 3-20, 3-19, 3-18, 3-17, 3-16, 3-15, 3-14, 3-13, 3-12, 3-11, 3-10, 3-9, 3-8, 3-7, 4-28, 4-27, 4-26, 4-25, 4-24, 4-23, 4-22, 4-21, 4-20, 4-19, 4-18, 4-17, 4-16, 4-15, 4-14, 4-13, 4-12, 4-11, 4-10, 4-9, 4-8, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 5-9, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 6-10, 7-28, 7-27, 7-26, 7-25, 7-24, 7-23, 7-22, 7-21, 7-20, 7-19, 7-18, 7-17, 7-16, 7-15, 7-14, 7-13, 7-12, 7-11, 8-28, 8-27, 8-26, 8-25, 8-24, 8-23, 8-22, 8-21, 8-20, 8-19, 8-18, 8-17, 8-16, 8-15, 8-14, 8-13, 8-12, 9-28, 9-27, 9-26, 9-25, 9-24, 9-23, 9-22, 9-21, 9-20, 9-19, 9-18, 9-17, 9-16, 9-15, 9-14, 9-13, 10-28, 10-27, 10-26, 10-25, 10-24, 10-23, 10-22, 10-21, 10-20, 10-19, 10-18, 10-17, 10-16, 10-15, 10-14, 11-28, 11-27, 11-26, 11-25, 11-24, 11-23, 11-22, 11-21, 11-20, 11-19, 11-18, 11-17, 11-16, 11-15, 12-28, 12-27, 12-26, 12-25, 12-24, 12-23, 12-22, 12-21, 12-20, 12-19, 12-18, 12-17, 12-16, 13-28, 13-27, 13-26, 13-25, 13-24, 13-23, 13-22, 13-21, 13-20, 13-19, 13-18, 13-17, 14-28, 14-27, 14-26, 14-25, 14-24, 14-23, 14-22, 14-21, 14-20, 14-19, 14-18, 15-28, 15-27, 15-26, 15-25, 15-24, 15-23, 15-22, 15-21, 15-20, 15-19, 16-32, 16-31, 16-30, 16-29, 16-28, 16-27, 16-26, 16-25, 16-24, 16-23, 16-22, 16-21, 16-20, 17-28, 17-27, 17-26, 17-25, 17-24, 17-23, 17-22, 17-21, 18-28, 18-27, 18-26, 18-25, 18-24, 18-23, 18-22, 19-28, 19-27, 19-26, 19-25, 19-24, 19-23, 20-28, 20-27, 20-26, 20-25, 20-24, -28, 21-27, 21-26, 21-25, 22-32, 22-31, 22-30, 22-29, 22-28, 22-27, 22-26, 23-28 or 23-27 of SEQ ID NO:8.

h) Human B-Type Natriuretic Peptide (hBNP) and Fragment

As used herein, the phrases “human B-Type natriuretic peptide”, “human BNP”, “hBNP”, “hBNP-32”, “hBNP peptide”, “hBNP polypeptide”, or “B-type natriuretic peptide” used interchangeably herein, refer to a 32 amino acid molecule having the amino acid sequence shown in SEQ ID NO:4. The amino acid sequence shown in SEQ ID NO:4 is represented by amino acids 77-108 of the 108 amino acid sequence of human proBNP (SEQ ID NO:2).

As used herein, the phrases “human B-type natriuretic peptide fragment”, “hBNP fragment” “hBNP-32 fragment” or “hBNP peptide fragment” as used interchangeably herein refers to a polypeptide that comprises a fragment of a hBNP peptide that contains a contiguous or nonlinear epitope of the hBNP peptide. The precise boundaries of such an epitope fragment can be confirmed using ordinary skill in the art. In one aspect, a hBNP fragment or hBNP peptide fragment refers to a peptide that comprises at least five contiguous amino acids of SEQ ID NO:4. In another aspect, a hBNP fragment or hBNP peptide fragment refers to a peptide that comprises at least ten contiguous amino acids residues of SEQ ID NO:4; at least fifteen contiguous amino acids residues of amino acids of SEQ ID NO:4; at least 20 contiguous amino acids residues of SEQ ID NO:4; at least 25 contiguous amino acids residues of SEQ ID NO:4 or at least 30 contiguous amino acid residues of amino acids of SEQ ID NO:4. Examples of hBNP fragments or hBNP peptide fragments include, but are not limited to, amino acid sequences containing amino acids residues 1-31, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 1-9, 1-8, 1-7, 1-6, 1-5, 2-32, 2-31, 2-30, 2-29, 2-28, 2-27, 2-26, 2-25, 2-24, 2-23, 2-22, 2-21, 2-20, 2-19, 2-18, 2-17, 2-16, 2-15, 2-14, 2-13, 2-12, 2-11, 2-10, 2-9, 2-8, 2-7, 2-6, 3-32, 3-31, 3-30, 3-29, 3-28, 3-27, 3-26, 3-25, 3-24, 3-23, 3-22, 3-21, 3-20, 3-19, 3-18, 3-17, 3-16, 3-15, 3-14, 3-13, 3-12, 3-11, 3-10, 3-9, 3-8, 3-7, 4-32, 4-31, 4-30, 4-29, 4-28, 4-27, 4-26, 4-25, 4-24, 4-23, 4-22, 4-21, 4-20, 4-19, 4-18, 4-17, 4-16, 4-15, 4-14, 4-13, 4-12, 4-11, 4-10, 4-9, 4-8, 5-32, 5-31, 5-30, 5-29, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 5-9, 6-32, 6-31, 6-30, 6-29, 6-28, 6-27, 6-26, 6-25, 6-24, 6-23, 6-22, 6-21, 6-20, 6-19, 6-18, 6-17, 6-16, 6-15, 6-14, 6-13, 6-12, 6-11, 6-10, 7-32, 7-31, 7-30, 7-29, 7-28, 7-27, 7-26, 7-25, 7-24, 7-23, 7-22, 7-21, 7-20, 7-19, 7-18, 7-17, 7-16, 7-15, 7-14, 7-13, 7-12, 7-11, 8-32, 8-31, 8-30, 8-29, 8-28, 8-27, 8-26, 8-25, 8-24, 8-23, 8-22, 8-21, 8-20, 8-19, 8-18, 8-17, 8-16, 8-15, 8-14, 8-13, 8-12, 9-32, 9-31, 9-30, 9-29, 9-28, 9-27, 9-26, 9-25, 9-24, 9-23, 9-22, 9-21, 9-20, 9-19, 9-18, 9-17, 9-16, 9-15, 9-14, 9-13, 10-32, 10-31, 10-30, 10-29, 10-28, 10-27, 10-26, 10-25, 10-24, 10-23, 10-22, 10-21, 10-20, 10-19, 10-18, 10-17, 10-16, 10-15, 10-14, 11-32, 11-31, 11-30, 11-29, 11-28, 11-27, 11-26, 11-25, 11-24, 11-23, 11-22, 11-21, 11-20, 11-19, 11-18, 11-17, 11-16, 11-15, 12-32, 12-31, 12-30, 12-29, 12-28, 12-27, 12-26, 12-25, 12-24, 12-23, 12-22, 12-21, 12-20, 12-19, 12-18, 12-17, 12-16, 13-32, 13-31, 13-30, 13-29, 13-28, 13-27, 13-26, 13-25, 13-24, 13-23, 13-22, 13-21, 13-20, 13-19, 13-18, 13-17, 14-32, 14-31, 14-30, 14-29, 14-28, 14-27, 14-26, 14-25, 14-24, 14-23, 14-22, 14-21, 14-20, 14-19, 14-18, 15-32, 15-31, 15-30, 15-29, 15-28, 15-27, 15-26, 15-25, 15-24, 15-23, 15-22, 15-21, 15-20, 15-19, 16-32, 16-31, 16-30, 16-29, 16-28, 16-27, 16-26, 16-25, 16-24, 16-23, 16-22, 16-21, 16-20, 17-32, 17-31, 17-30, 17-29, 17-28, 17-27, 17-26, 17-25, 17-24, 17-23, 17-22, 17-21, 18-32, 18-31, 18-30, 18-29, 18-28, 18-27, 18-26, 18-25, 18-24, 18-23, 19-22, 19-32, 19-31, 19-30, 19-29, 19-28, 19-27, 19-26, 19-25, 19-24, 19-23, 20-32, 20-31, 20-30, 20-29, 20-28, 20-27, 20-26, 20-25, 20-24, 21-32, 21-31, 21-30, 21-29, 21-28, 21-27, 21-26, 21-25, 22-32, 22-31, 22-30, 22-29, 22-28, 22-27, 22-26, 23-32, 23-31, 23-30, 23-29, 23-28, 23-27, 24-32, 24-31, 24-30, 24-29, 24-28, 25-32, 25-31, 25-30, 25-29, 26-32, 26-31, 26-30, 27-32 or 27-31 of SEQ ID NO:4.

i) Human C-Type Natriuretic Peptide (hCNP) and Fragment

As used herein, the phrases “human C-type natriuretic peptide”, “human CNP”, “hCNP”, “hCNP peptide”, or “hCNP polypeptide” as used interchangeably herein, refer to a 22 amino acid molecule having the amino acid sequence shown in SEQ ID NO:13.

As used herein, the phrases “human C-type natriuretic peptide fragment”, “hCNP fragment” or “hCNP peptide fragment” as used interchangeably herein, refer to a polypeptide that comprises a fragment of an hCNP peptide that contains a contiguous or nonlinear epitope of the hCNP peptide. The precise boundaries of such an epitope fragment can be confirmed using ordinary skill in the art. In one aspect, an hCNP fragment or hCNP peptide fragment refers to a peptide that comprises at least five contiguous amino acids of SEQ ID NO:13. In another aspect, a hCNP fragment or hCNP peptide fragment refers to a peptide that comprises at least ten contiguous amino acids residues of SEQ ID NO:13; at least fifteen contiguous amino acids residues of amino acids of SEQ ID NO:13 or at least 18 contiguous amino acids residues of SEQ ID NO:13. Examples of hCNP fragments or hCNP peptide fragments include, but are not limited to, amino acid sequences containing amino acids residues 1-21, 1-20, 1-19, 1-18, 1-17, 1-16, 1-15, 1-14, 1-13, 1-12, 1-11, 1-10, 1-9, 1-8, 1-7, 1-6, 1-5, 2-22, 2-21, 2-20, 2-19, 2-18, 2-17, 2-16, 2-15, 2-14, 2-13, 2-12, 2-11, 2-10, 2-9, 2-8, 2-7, 2-6, 3-22, 3-21, 3-20, 3-19, 3-18, 3-17, 3-16, 3-15, 3-14, 3-13, 3-12, 3-11, 3-10, 3-9, 3-8, 3-7, 4-22, 4-21, 4-20, 4-19, 4-18, 4-17, 4-16, 4-15, 4-14, 4-13, 4-12, 4-11, 4-10, 4-9, 4-8, 5-22, 5-21, 5-20, 5-19, 5-18, 5-17, 5-16, 5-15, 5-14, 5-13, 5-12, 5-11, 5-10, 5-9, 6-22, 6-21, 6-20, 6-19, 6-18, 6-17, 6-16, 6-15, 6-14, 6-13, 6-12, 6-11, 6-10, 7-22, 7-21, 7-20, 7-19, 7-18, 7-17, 7-16, 7-15, 7-14, 7-8-22, 8-21, 8-20, 8-19, 8-18, 8-17, 8-16, 8-15, 8-14, 8-13, 8-12, 9-22, 9-21, 9-20, 9-19, 9-18, 9-17, 9-16, 9-15, 9-14, 9-13, 10-22, 10-21, 10-20, 10-19, 10-18, 10-17, 10-16, 10-15, 10-14, 11-22, 11-21, 11-20, 11-19, 11-18, 11-17, 11-16, 11-15, 12-22, 12-21, 12-20, 12-19, 12-18, 12-17, 12-16, 13-22, 13-21, 13-20, 13-19, 13-18, 13-17, 14-22, 14-21, 14-20, 14-19, 14-18, 15-22, 15-21, 15-20, 15-19, 16-22, 16-21, 16-20, 17-22, 17-21 or 18-22 of SEQ ID NO:13.

j) Human A-Type Natriuretic proPeptide (proANP) and Fragment

As used herein, the phrases “human A-Type natriuretic pro peptide”, “pro peptide of human ANP”, “human proANP”, “h-proANP”, “h-proANP-126”, “h-proANP peptide”, “h-proANP polypeptide”, or “A-type natriuretic propeptide” used interchangeably herein, refer to a 126 amino acid molecule having the amino acid sequence shown in SEQ ID NO:6. Human proANP is derived from human pre-pro ANP.

As used herein, the phrases “human A-type natriuretic pro peptide fragment”, “h-proANP fragment” “h-proANP-126 fragment” or “h-proANP peptide fragment” as used interchangeably herein refers to a polypeptide that comprises a fragment of a proANP peptide. In one aspect, the proANP fragments comprise hANP having the amino acid sequence shown in SEQ ID NO:8 or NT-proANP having the amino acid sequence shown in SEQ ID NO:7. In another aspect, the fragment of a proANP peptide contains a contiguous or nonlinear epitope of the h-proANP peptide. The precise boundaries of such an epitope fragment can be confirmed using ordinary skill in the art. In one aspect, a proANP fragment or proANP peptide fragment refers to a peptide that comprises at least five contiguous amino acids of SEQ ID NO:6. In another aspect, a proANP fragment or proANP peptide fragment refers to a peptide that comprises at least ten contiguous amino acids residues of SEQ ID NO:6; at least fifteen contiguous amino acids residues of amino acids of SEQ ID NO:6; at least 20 contiguous amino acids residues of SEQ ID NO:6; at least 25 contiguous amino acids residues of SEQ ID NO:6 or at least 30 contiguous amino acid residues of amino acids of SEQ ID NO:6. Examples of proANP fragments or proBNP peptide fragments include, but are not limited to, amino acid sequences containing amino acids residues 1-98, 124-153, 124-152, 124-151, 124-150, 124-149, 124-148, 124-147, 124-146, 124-145, 124-144, 124-143, 124-142, 124-141, 124-140, 124-139, 124-138, 124-137, 124-136, 124-135, 124-134, 124-133, 124-132, 124-131, 124-130, 124-129, 124-128, 125-151, 125-150, 125-149, 125-148, 125-147, 125-146, 125-145, 125-144, 125-143, 125-142, 125-141, 125-140, 125-139, 125-138, 125-137, 125-136, 125-135, 125-134, 125-133, 125-132, 125-131, 125-130, 125-129, 126-151, 126-150, 126-149, 126-148, 126-147, 126-146, 126-145, 126-144, 126-143, 126-142, 126-141, 126-140, 126-139, 126-138, 126-137, 126-136, 126-135, 126-134, 126-133, 126-132, 126-131, 126-130, 127-151, 127-150, 127-149, 127-148, 127-147, 127-146, 127-145, 127-144, 127-143, 127-142, 127-141, 127-140, 127-139, 127-138, 127-137, 127-136, 127-135, 127-134, 127-133, 127-132, 127-131, 128-151, 128-150, 128-149, 128-148, 128-147, 128-146, 128-145, 128-144, 128-143, 128-142, 128-141, 128-140, 128-139, 128-138, 128-137, 128-136, 128-135, 128-134, 128-133, 128-132, 129-151, 129-150, 129-149, 129-148, 129-147, 129-146, 129-145, 129-144, 129-143, 129-142, 129-141, 129-140, 129-139, 129-138, 129-137, 129-136, 129-135, 129-134, 129-133, 130-151, 130-150, 130-149, 130-148, 130-147, 130-146, 130-145, 130-144, 130-143, 130-142, 130-141, 130-140, 130-139, 130-138, 130-137, 130-136, 130-135, 130-134, 131-151, 131-150, 131-149, 131-148, 131-147, 131-146, 131-145, 131-144, 131-143, 131-142, 131-141, 131-140, 131-139, 131-138, 131-137, 131-136, 131-135, 132-151, 132-150, 132-149, 132-148, 132-147, 132-146, 132-145, 132-144, 132-143, 132-142, 132-141, 132-140, 132-139, 132-138, 132-137, 132-136, 133-151, 133-150, 133-149, 133-148, 133-147, 133-146, 133-145, 133-144, 133-143, 133-142, 133-141, 133-140, 133-139, 133-138, 133-137, 134-151, 134-150, 134-149, 134-148, 134-147, 134-146, 134-145, 134-144, 134-143, 134-142, 134-141, 134-140, 134-139, 134-138, 135-151, 135-150, 135-149, 135-148, 135-147, 135-146, 135-145, 135-144, 135-143, 135-142, 135-141, 135-140, 135-139, 136-151, 136-150, 136-149, 136-148, 136-147, 136-146, 136-145, 136-144, 136-143, 136-142, 136-141, 136-140, 137-151, 137-150, 137-149, 137-148, 137-147, 137-146, 137-145, 137-144, 137-143, 137-142, 137-141, 138-151, 138-150, 138-149, 138-148, 138-147, 138-146, 138-145, 138-144, 138-143, 138-142, 139-151, 139-150, 139-149, 139-148, 139-147, 139-146, 139-145, 139-144, 139-143, 140-151, 140-150, 140-149, 140-148, 140-147, 140-146, 140-145, 140-144, 141-151, 141-150, 141-149, 141-148, 141-147, 141-146, 141-145, 142-151, 142-150, 142-149, 142-148, 142-147, 142-146, 143-151, 143-150, 143-149, 143-148, 143-147, 144-151, 144-150, 144-149, 144-148, 145-151, 145-150, 145-149, 146-151, 146-150 or 147-151 of SEQ ID NO:6.

k) Human B-Type Natriuretic proPeptide (proBNP) and Fragment

As used herein, the phrases “human B-Type natriuretic propeptide”, “pro peptide of human BNP”, “human proBNP”, “h-proBNP”, “h-proBNP-108”, “h-proBNP peptide”, “h-proBNP polypeptide”, or “B-type natriuretic propeptide” used interchangeably herein, refer to a 108 amino acid molecule having the amino acid sequence shown in SEQ ID NO:2. Human proBNP is derived from human pre-pro BNP.

As used herein, the phrases “human B-type natriuretic pro peptide fragment”, “h-proBNP fragment” “h-proBNP-108 fragment” or “h-proBNP peptide fragment” as used interchangeably herein refers to a polypeptide that comprises a fragment of a proBNP peptide. In one aspect, the proBNP fragments comprise hBNP having the amino acid sequence shown in SEQ ID NO:4 or NT-proBNP having the amino acid sequence shown in SEQ ID NO: 3. In another aspect, the fragment of a proBNP peptide contains a contiguous or nonlinear epitope of the h-proBNP peptide. The precise boundaries of such an epitope fragment can be confirmed using ordinary skill in the art. In one aspect, a proBNP fragment or proBNP peptide fragment refers to a peptide that comprises at least five contiguous amino acids of SEQ ID NO:2. In another aspect, a proBNP fragment or proBNP peptide fragment refers to a peptide that comprises at least ten contiguous amino acids residues of SEQ ID NO:2; at least fifteen contiguous amino acids residues of amino acids of SEQ ID NO:2; at least 20 contiguous amino acids residues of SEQ ID NO:2; at least 25 contiguous amino acids residues of SEQ ID NO:2 or at least 30 contiguous amino acid residues of amino acids of SEQ ID NO:2. Examples of proBNP fragments or proBNP peptide fragments include, but are not limited to, amino acid sequences containing amino acids residues 1-76, 77-108, 1-30, 2-31, 3-32, 4-33, 5-34, 6-35, 7-36, 8-37, 9-38, 10-39, 11-40, 12-41, 13-42, 14-43, 15-44, 16-45, 17-46, 18-47, 19-48, 20-49, 21-50, 22-51, 23-52, 24-53, 25-54, 26-55, 27-56, 28-57, 29-58, 30-59, 31-60, 32-61, 33-62, 34-63, 35-64, 36-65, 37-66, 38-67, 39-68, 40-69, 41-70, 42-71, 43-72, 44-73, 45-74, 46-75, 47-76, 48-77, 49-78, 50-79, 51-80, 52-81, 53-82, 54-83, 55-84, 56-85, 57-86, 58-87, 59-88, 60-89, 61-90, 62-91, 63-92, 64-93, 65-94, 66-95, 67-96, 68-97, 69-98, 70-99, 71-100, 72-101, 73-102, 74-103, 75-104, 76-105, 77-106, 78-107, 79-108, 1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100, 82-101, 83-102, 84-103, 85-104, 86-105, 87-106, 88-107, 89-108, 1-15, 2-16, 3-17, 4-18, 5-19, 6-20, 7-21, 8-22, 9-23, 10-24, 11-25, 12-26, 13-27, 14-28, 15-29, 16-30, 17-31, 18-32, 19-33, 20-34, 21-35, 22-36, 23-37, 24-38, 25-39, 26-40, 27-41, 28-42, 29-43, 30-44, 31-45, 32-46, 33-47, 34-48, 35-49, 36-50, 37-51, 38-52, 39-53, 40-54, 41-55, 42-56, 43-57, 44-58, 45-59, 46-60, 47-61, 48-62, 49-63, 50-64, 51-65, 52-66, 53-67, 54-68, 55-69, 56-70, 57-71, 58-72, 59-73, 60-74, 61-75, 62-76, 63-77, 64-78, 65-79, 66-80, 67-81, 68-82, 69-83, 70-84, 71-85, 72-86, 73-87, 74-88, 75-89, 76-90, 77-91, 78-92, 79-93, 80-94, 81-95, 82-96, 83-97, 84-98, 85-99, 86-100, 87-101, 88-102, 89-103, 90-104, 91-105, 92-106, 93-107, 94-108, 1-10, 2-11, 3-12, 4-13, 5-14, 6-15, 7-16, 8-17, 9-18, 10-19, 11-20, 12-21, 13-22, 14-23, 15-24, 16-25, 17-26, 18-27, 19-28, 20-29, 21-30, 22-31, 23-32, 24-33, 25-34, 26-35, 27-36, 28-37, 29-38, 30-39, 31-40, 32-41, 33-42, 34-43, 35-44, 36-45, 37-46, 38-47, 39-48, 40-49, 41-50, 42-51, 43-52, 44-53, 45-54, 46-55, 47-56, 48-57, 49-58, 50-59, 51-60, 52-61, 53-62, 54-63, 55-64, 56-65, 57-66, 58-67, 59-68, 60-69, 61-70, 62-71, 63-72, 64-73, 65-74, 66-75, 67-76, 68-77, 69-78, 70-79, 71-80, 72-81, 73-82, 74-83, 75-84, 76-85, 77-86, 78-87, 79-88, 80-89, 81-90, 82-91, 83-92, 84-93, 85-94, 86-95, 87-96, 88-97, 89-98, 90-99, 91-100, 92-101, 93-102, 94-103, 95-104, 96-105, 97-106, 98-107, 99-108 1-5, 2-6, 3-7, 4-8, 5-9, 6-10, 7-11, 8-12, 9-13, 10-14, 11-15, 12-16, 13-17, 14-18, 15-19, 16-20, 17-21, 18-22, 19-23, 20-24, 21-25, 22-26, 23-27, 24-28, 25-29, 26-30, 27-31, 28-32, 29-33, 30-34, 31-35, 32-36, 33-37, 34-38, 35-39, 36-40, 37-41, 38-42, 39-43, 40-44, 41-45, 42-46, 43-47, 44-48, 45-49, 46-50, 47-51, 48-52, 49-53, 50-54, 51-55, 52-56, 53-57, 54-58, 55-59, 56-60, 57-61, 58-62, 59-63, 60-64, 61-65, 62-66, 63-67, 64-68, 65-69, 66-70, 67-71, 68-72, 69-73, 70-74, 71-75, 72-76, 73-77, 74-78, 75-79, 76-80, 77-81, 78-82, 79-83, 80-84, 81-85, 82-86, 83-87, 84-88, 85-89, 86-90, 87-91, 88-92, 89-93, 90-94, 91-95, 92-96, 93-97, 94-98, 95-99, 96-100, 97-101, 98-102, 99-103, 100-104, 101-105, 102-106, 103-107 or 104-108 of SEQ ID NO:2.

l) Human C-Type Natriuretic proPeptide (proCNP) and Fragment

As used herein, the phrases “human C-Type natriuretic propeptide”, ““pro peptide of human CNP”, “human proCNP”, “h-proCNP”, “h-proCNP-103”, “h-proCNP peptide”, “h-proCNP polypeptide”, or “C-type natriuretic propeptide” used interchangeably herein, refer to a 103 amino acid molecule having the amino acid sequence shown in SEQ ID NO:10. Human proCNP is derived from human pre-pro CNP.

As used herein, the phrases “human C-type natriuretic pro peptide fragment”, “h-proCNP fragment” “h-proCNP-103 fragment” or “h-proCNP peptide fragment” as used interchangeably herein refers to a polypeptide that comprises a fragment of a proCNP peptide. In one aspect, the proCNP fragments comprise hCNP having the amino acid sequence shown in SEQ ID NO:13 or NT-proCNP having the amino acid sequence shown in SEQ ID NO: 15, hCNP-53 having the amino acid sequence shown in SEQ ID NO:11 or hCNP-29 polypeptide having the amino acid sequence shown in SEQ ID NO:12.

In another aspect, the fragment of a proCNP peptide contains a contiguous or nonlinear epitope of the h-proCNP peptide. The precise boundaries of such an epitope fragment can be confirmed using ordinary skill in the art. In one aspect, a proCNP fragment or proCNP peptide fragment refers to a peptide that comprises at least five contiguous amino acids of SEQ ID NO:10. In another aspect, a proCNP fragment or proCNP peptide fragment refers to a peptide that comprises at least ten contiguous amino acids residues of SEQ ID NO:10; at least fifteen contiguous amino acids residues of amino acids of SEQ ID NO:10; at least 20 contiguous amino acids residues of SEQ ID NO:10; at least 25 contiguous amino acids residues of SEQ ID NO:10 or at least 30 contiguous amino acid residues of amino acids of SEQ ID NO:10. Examples of proCNP fragments or proCNP peptide fragments include, but are not limited to, amino acid sequences containing amino acids residues 1-50, 51-103, 75-103, 82-103, 82-102, 82-101, 82-100, 82-99, 82-98, 82-97, 82-96, 82-95, 82-94, 82-93, 82-92, 82-91, 82-90, 82-89, 82-88, 82-87, 82-86, 83-103, 83-102, 83-101, 83-100, 83-99, 83-98, 83-97, 83-96, 83-95, 83-94, 83-93, 83-92, 83-91, 83-90, 83-89, 83-88, 83-87, 84-103, 84-102, 84-101, 84-100, 84-99, 84-98, 84-97, 84-96, 84-95, 84-94, 84-93, 84-92, 84-91, 84-90, 84-89, 84-88, 85-103, 85-102, 85-101, 85-100, 85-99, 85-98, 85-97, 85-96, 85-95, 85-94, 85-93, 85-92, 85-91, 85-90, 85-89, 86-103, 86-102, 86-101, 86-100, 86-99, 86-98, 86-97, 86-96, 86-95, 86-94, 86-93, 86-92, 86-91, 86-90, 87-103, 87-101, 87-100, 87-99, 87-98, 87-97, 87-96, 87-95, 87-94, 87-93, 87-92, 87-91, 88-103, 88-102, 88-101, 88-100, 88-99, 88-98, 88-97, 88-96, 88-95, 88-94, 88-93, 88-92, 89-103, 89-102, 89-101, 89-100, 89-99, 89-98, 89-97, 89-96, 89-95, 89-94, 89-93, 90-103, 90-102, 90-101, 90-100, 90-99, 90-98, 90-97, 90-96, 90-95, 90-94, 91-103, 91-102, 91-101, 91-100, 91-99, 91-98, 91-97, 91-96, 91-95, 92-103, 92-102, 92-101, 92-100, 92-99, 92-98, 92-97, 92-96, 93-103, 93-102, 93-101, 93-100, 93-99, 93-98, 93-97, 94-103, 94-102, 94-101, 94-100, 94-99, 94-98, 95-103, 95-102, 95-101, 95-100, 95-99, 96-103, 96-102, 96-101, 96-100, 97-103, 97-102, 97-101, 98-103, 98-102 or 99-103 of SEQ ID NO:10.

m) Human Natriuretic Peptide and Fragment

As used herein, the phrase “human natriuretic peptide” refers to pre-pro peptide precursor of human ANP, pro peptide of human ANP, N-terminal pro peptide of ANP, human ANP, pre-pro peptide precursor of human BNP, pro peptide of human BNP, N-terminal pro peptide of BNP, human BNP, human CNP, pro peptide of human CNP or any combinations thereof.

As used herein, the phrase “human natriuretic peptide fragment” refers to human A-type pro peptide fragment, human A-type natriuretic peptide fragment, human B-type pro peptide fragment, human B-type natriuretic peptide fragment, human C-type pro peptide fragment, human C-type natriuretic peptide fragment or any combinations thereof

n) Human Natriuretic Peptide Analog

As used herein, the phrase “human natriuretic peptide analog” refers to a biologically active analog of a human natriuretic peptide (e.g., human BNP). For example, a biologically active human natriuretic peptide analog can be a human natriuretic peptide with truncations, deletions, insertions, substitutions, replacements, side chain extensions, and fusion proteins, or combinations of the foregoing which do not eliminate the biological activity of the original compound. Human natriuretic peptide analogs can be obtained by various means. For example, certain amino acids can be substituted for other amino acids in the native natriuretic peptide structure without eliminating interactive binding capacity. Examples of human natriuretic peptide analogs and methods for making such analogs are described in U.S. Patent Application No. 2006/0172933.

o) Human Natriuretic Peptide Conjugate

As used herein, the phrase “human natriuretic peptide conjugate” refers to human natriuretic peptide or human natriuretic peptide fragment that includes at least one modifying moiety or at least one reactive entity attached thereto. Modifying moieties are moieties that modify a human natriuretic peptide or a human natriuretic peptide fragment (e.g., hBNP or hBNP fragment). Example of modifying moieties include, but are not limited to, moieties that effect stability, solubility, and/or biological activity (e.g., hydrophilic polymers or oligomers, amphiphilic polymers or oligomers, and lipophilic polymers or oligomers), hydrophilic moieties, polyethylene glycol moieties, biocompatible water soluble moieties, polycationic moieties, amphiphilic moieties, polyethylene glycol/alkyl modifying moieties, etc. (each of which are described in U.S. Patent Application No. 2006/0172933).

Human natriuretic peptides or human natriuretic peptide fragments can be chemically modified (by covalent bonding) by coupling to a reactive entity as described in U.S. Patent Application No. 2004/0266673. The reactive entity is capable of forming a covalent bond with a blood component, preferably a blood protein. The covalent bond is generally formed between the reactive entity and an amino group, a hydroxyl group, or a thiol group on the blood component. The amino group can form a covalent bond with reactive entities like carboxy, phosphoryl or acyl; the hydroxyl group preferably forms a covalent bond with reactive entities like activated esters; and the thiol group preferably forms a covalent bond with reactive entities like esters or mixed anhydrides. The preferred blood components are mobile blood components like serum albumin, immunoglobulins, or combinations thereof, and the preferred reactive entity comprises anhydrides like maleimide or maleimido-containing groups.

Methods for conjugating a modifying moiety to a base molecules, such as a human natriuretic peptides (e.g., human BNP) are well known in the art. For example, strategies for conjugating a modifying moiety to human natriuretic peptide are disclosed in U.S. Patent Application No. 2006/0172933. Methods for chemically modifying human natriuretic peptides to a reactive entity are described in U.S. Patent Application No. 2004/0266673.

p) Human Natriuretic Peptide Derivative

As used herein, the phrase “human natriuretic peptide derivative” refers to a human natriuretic peptide analog, a human natriuretic peptide conjugate or a recombinant form of a human natriuretic peptide (e.g., a recombinant form of human BNP (SEQ ID NO:4) (e.g, nesiritide)).

q) Hydrogen Peroxide Generating Enzyme

As used herein, the term “hydrogen peroxide generating enzyme” refers to an enzyme that is capable of generating hydrogen peroxide. Examples of hydrogen peroxide generating enzymes are listed below in Table 1.

TABLE 1

| | IUBMB ENZYME | PREFERRED |
|---|---|---|
| ACCEPTED COMMON NAME | NOMENCLATURE | SUBSTRATE |
| (R)-6-hydroxynicotine oxidase | EC 1.5.3.6 | (R)-6-hydroxynicotine |
| (S)-2-hydroxy acid oxidase | EC 1.1.3.15 | S)-2-hydroxy acid |
| (S)-6-hydroxynicotine oxidase | EC 1.5.3.5 | (S)-6-hydroxynicotine |
| 3-aci-nitropropanoate oxidase | EC 1.7.3.5 | 3-aci-nitropropanoate |
| 3-hydroxyanthranilate oxidase | EC 1.10.3.5 | 3-hydroxyanthranilate |
| 4-hydroxymandelate oxidase | EC 1.1.3.19 | (S)-2-hydroxy- |
| | | 2-(4-hydroxyphenyl)acetate |
| 6-hydroxynicotinate dehydrogenase | EC 1.17.3.3 | 6-hydroxynicotinate |
| Abscisic-aldehyde oxidase | EC 1.2.3.14 | abscisic aldehyde |
| acyl-CoA oxidase | EC 1.3.3.6 | acyl-CoA |
| Alcohol oxidase | EC 1.1.3.13 | a primary alcohol |
| Aldehyde oxidase | EC 1.2.3.1 | an aldehyde |
| amine oxidase | | |
| amine oxidase (copper-containing) | EC 1.4.3.6 | primary monoamines, |
| | | diamines and histamine |
| amine oxidase (flavin-containing) | EC 1.4.3.4 | a primary amine |
| aryl-alcohol oxidase | EC 1.1.3.7 | an aromatic primary |
| | | alcohol |
| | | (2-naphthyl)methanol |
| | | 3-methoxybenzyl alcohol |
| aryl-aldehyde oxidase | EC 1.2.3.9 | an aromatic aldehyde |
| Catechol oxidase | EC 1.1.3.14 | Catechol |
| Cholesterol oxidase | EC 1.1.3.6 | Cholesterol |
| Choline oxidase | EC 1.1.3.17 | Choline |
| columbamine oxidase | EC 1.21.3.2 | Columbamine |
| cyclohexylamine oxidase | EC 1.4.3.12 | Cyclohexylamine |
| cytochrome c oxidase | EC 1.9.3.1 | |
| D-amino-acid oxidase | EC 1.4.3.3 | a D-amino acid |
| D-arabinono-1,4-lactone oxidase | EC 1.1.3.37 | D-arabinono-1,4-lactone |
| D-arabinono-1,4-lactone oxidase | EC 1.1.3.37 | D-arabinono-1,4-lactone |
| D-aspartate oxidase | EC 1.4.3.1 | D-aspartate |
| D-glutamate oxidase | EC 1.4.3.7 | D-glutamate |
| D-glutamate(D-aspartate) oxidase | EC 1.4.3.15 | D-glutamate |
| dihydrobenzophenanthridine | EC 1.5.3.12 | dihydrosanguinarine |
| oxidase | | |
| dihydroorotate oxidase | EC 1.3.3.1 | (S)-dihydroorotate |
| dihydrouracil oxidase | EC 1.3.3.7 | 5,6-dihydrouracil |
| dimethylglycine oxidase | EC 1.5.3.10 | N,N-dimethylglycine |
| D-mannitol oxidase | EC 1.1.3.40 | Mannitol |
| Ecdysone oxidase | EC 1.1.3.16 | Ecdysone |
| ethanolamine oxidase | EC 1.4.3.8 | Ethanolamine |
| Galactose oxidase | EC 1.1.3.9 | D-galactose |
| Glucose oxidase | EC 1.1.3.4 | β-D-glucose |
| Glutathione oxidase | EC 1.8.3.3 | Glutathione |
| glycerol-3-phosphate oxidase | EC 1.1.3.21 | sn-glycerol 3-phosphate |
| Glycine oxidase | EC 1.4.3.19 | Glycine |
| glyoxylate oxidase | EC 1.2.3.5 | Glyoxylate |
| hexose oxidase | EC 1.1.3.5 | D-glucose, |
| | | D-galactose |
| | | D-mannose |
| | | maltose |
| | | lactose |
| | | cellobiose |
| hydroxyphytanate oxidase | EC 1.1.3.27 | L-2-hydroxyphytanate |
| indole-3-acetaldehyde oxidase | EC 1.2.3.7 | (indol-3-yl)acetaldehyde |
| lactic acid oxidase | | Lactic acid |
| L-amino-acid oxidase | EC 1.4.3.2 | an L-amino acid |
| L-aspartate oxidase | EC 1.4.3.16 | L-aspartate |
| L-galactonolactone oxidase | EC 1.3.3.12 | L-galactono-1,4-lactone |
| L-glutamate oxidase | EC 1.4.3.11 | L-glutamate |
| L-gulonolactone oxidase | EC 1.1.3.8 | L-gulono-1,4-lactone |
| L-lysine 6-oxidase | EC 1.4.3.20 | L-lysine |
| L-lysine oxidase | EC 1.4.3.14 | L-lysine |
| long-chain-alcohol oxidase | EC 1.1.3.20 | A long-chain-alcohol |
| L-pipecolate oxidase | EC 1.5.3.7 | L-pipecolate |
| L-sorbose oxidase | EC 1.1.3.11 | L-sorbose |
| malate oxidase | EC 1.1.3.3 | (S)-malate |
| methanethiol oxidase | EC 1.8.3.4 | Methanethiol |
| monoamino acid oxidase | | |
| N⁶-methyl-lysine oxidase | EC 1.5.3.4 | 6-N-methyl-L-lysine |
| N-acylhexosamine oxidase | EC 1.1.3.29 | N-acetyl-D-glucosamine |
| | | N-glycolylglucosamine |
| | | N-acetylgalactosamine |
| | | N-acetylmannosamine. |
| NAD(P)H oxidase | EC 1.6.3.1 | NAD(P)H |
| Nitroalkane oxidase | EC 1.7.3.1 | a nitroalkane |
| N-methyl-L-amino-acid oxidase | EC 1.5.3.2 | an N-methyl-L-amino |
| | | acid |
| nucleoside oxidase | EC 1.1.3.39 | Adenosine |
| oxalate oxidase | EC 1.2.3.4 | Oxalate |
| polyamine oxidase | EC 1.5.3.11 | 1-N-acetylspermine |
| Polyphenol oxidase | EC 1.14.18.1 | |
| Polyvinyl-alcohol oxidase | EC 1.1.3.30 | polyvinyl alcohol |
| prenylcysteine oxidase | EC 1.8.3.5 | an S-prenyl-L-cysteine |
| Protein-lysine 6-oxidase | EC 1.4.3.13 | peptidyl-L-lysyl-peptide |
| putrescine oxidase | EC 1.4.3.10 | butane-1,4-diamine |
| Pyranose oxidase | EC 1.1.3.10 | D-glucose |
| | | D-xylose |
| | | L-sorbose |
| | | D-glucono-1,5-lactone |
| Pyridoxal 5′-phosphate synthase | EC 1.4.3.5 | pyridoxamine 5′- |
| | | phosphate |
| pyridoxine 4-oxidase | EC 1.1.3.12 | Pyridoxine |
| pyrroloquinoline-quinone synthase | EC 1.3.3.11 | 6-(2-amino-2- |
| | | carboxyethyl)-7,8-dioxo- |
| | | 1,2,3,4,5,6,7,8- |
| | | octahydroquinoline-2,4- |
| | | dicarboxylate |
| Pyruvate oxidase | EC 1.2.3.3 | Pyruvate |
| Pyruvate oxidase (CoA-acetylating) | EC 1.2.3.6 | Pyruvate |
| Reticuline oxidase | EC 1.21.3.3 | Reticuline |
| retinal oxidase | EC 1.2.3.11 | Retinal |
| Rifamycin-B oxidase | EC 1.10.3.6 | rifamycin-B |
| Sarcosine oxidase | EC 1.5.3.1 | Sarcosine |
| secondary-alcohol oxidase | EC 1.1.3.18 | a secondary alcohol |
| sulfite oxidase | EC 1.8.3.1 | Sulfite |
| superoxide dismutase | EC 1.15.1.1 | Superoxide |
| superoxide reductase | EC 1.15.1.2 | Superoxide |
| tetrahydroberberine oxidase | EC 1.3.3.8 | (S)-tetrahydroberberine |
| Thiamine oxidase | EC 1.1.3.23 | Thiamine |
| tryptophan α,β-oxidase | EC 1.3.3.10 | L-tryptophan |
| urate oxidase (uricase, uric acid | EC 1.7.3.3 | uric acid |
| oxidase) | | |
| Vanillyl-alcohol oxidase | EC 1.1.3.38 | vanillyl alcohol |
| Xanthine oxidase | EC 1.17.3.2 | Xanthine |
| xylitol oxidase | EC 1.1.3.41 | Xylitol |

r) Isolated Human Natriuretic Peptide Autoantibody

As used herein, the phrase “isolated human natriuretic peptide autoantibody” refers to an antibody that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. Ordinarily, an isolated antibody will be prepared by at least one purification step.

s) Natriuretic Peptide and Fragment

As used herein, the term “natriuretic peptide” refers to at least one pre-pro peptide precursor of human ANP, pro peptide of human ANP, N-terminal pro peptide of ANP, human ANP, pre-pro peptide precursor of human BNP, pro peptide of human BNP, N-terminal pro peptide of BNP, human BNP, human CNP, pro peptide of human CNP, Dendroaspis natriuretic peptide and any combinations thereof.

As used herein, the term “natriuretic peptide fragment” refers to at least one human A-type pro peptide fragment, human A-type natriuretic peptide fragment, human B-type pro peptide fragment, human B-type natriuretic peptide fragment, human C-type pro peptide fragment, human C-type natriuretic peptide fragment, Dendroaspis natriuretic peptide fragment and any combinations thereof.

t) Pharmaceutical Composition

As used herein, the term “pharmaceutical composition” refers to any agent or drug, whether a small molecule (e.g., a drug containing an active agent, typically a non-peptidic) or biologic (e.g., a peptide or protein based drug, including any with modifications, such as, but not limited to PEGylation) that can be used to treat a subject suffering from a disease or condition that requires treatment. Examples of pharmaceutical compositions, include, but are not limited to, hyperlipidemia drugs (including, but not limited to, niacin, fibrates (e.g., clofibrate, fenofibrate, fenofibric acid, simfrate, salts of fenofibric acid and any combinations thereof), ezetimibe, HMG-CoA reductase inhibitors (e.g., statins, such as, but not limited to rosuvastatin, simvastatin, and combinations thereof (including combinations with other hyperlipidemia drugs (e.g., simvastatin and ezetimibe)), anti-inflammatories, natriuretic peptide and derivatives and analogs thereof (e.g., nesiritide, BNP, and combinations thereof), etc. as well as any combinations thereof.

u) Pre-Pro Peptide Precursors of Human ANP, Human BNP and Human CNP

As used herein, the term “pre-pro peptide precursor of human ANP” or “human pre-proANP” refers to a 153 amino acid molecule having the amino acid sequence shown in SEQ ID NO:5.

As used herein, the term “pre-pro peptide precursor of human BNP” or “human pre-proBNP” refers to a 134 amino acid molecule having the amino acid sequence shown in SEQ ID NO:1.

As used herein, the term “pre-pro peptide of human CNP” or “human pre-proCNP” refers to a 126 amino acid molecule having the amino acid sequence shown in SEQ ID NO:9.

v) Predetermined Level

As used herein, the term “predetermined level” refers generally at an assay cutoff value that is used to assess diagnostic results by comparing the assay results against the predetermined level, and where the predetermined level already that has been linked or associated with various clinical parameters (e.g., assessing risk, severity of disease, progression/nonprogression/improvement, etc.). The present disclosure provides exemplary predetermined levels, and describes the initial linkage or association of such levels with clinical parameters for exemplary immunoassays as described herein. However, it is well known that cutoff values may vary dependent on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this description.

w) Risk

As used herein, the term “risk” relates to the possibility or probability of a particular event occurring either presently, or, at some point in the future. “Risk stratification” refers to an arraying of known clinical risk factors to allow physicians to classify patients into a low, moderate, high or highest risk of developing of a particular disease, disorder or condition.

x) Specific Binding Partner

As used herein, the phrase “specific binding partner,” as used herein, is a member of a specific binding pair. That is, two different molecules where one of the molecules, through chemical or physical means, specifically binds to the second molecule. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzyme inhibitors, and enzymes and the like. Furthermore, specific binding pairs can include members that are analogs of the original specific binding members, for example, an analyte-analog. Immunoreactive specific binding members include antigens, antigen fragments, antibodies and antibody fragments, both monoclonal and polyclonal and complexes thereof, including those formed by recombinant DNA molecules.

y) Subject

A “subject” is a member of any animal species, preferably a mammalian species, optionally a human. The subject can be an apparently healthy individual, an individual suffering from a disease, and an individual being treated for a disease. A test subject is an individual from whom a reference sample is taken. A “reference subject” or “reference subjects” is/are an individual or a population that serves as a reference against which to assess another individual or population with respect to one or more parameters. Generally speaking, predetermined levels are obtained by examination and assessment of reference subjects.

Further with regard to such reference subjects, as described herein, “clinically normal cardiovascular function” means the reference subject has no known or apparent or presently detectable cardiovascular dysfunction and no detectable unfavorable aleration in levels (i.e., typically, an increase) in one or more autoantibodies reactive with human natriuretic peptide or a fragment thereof.

z) Test Sample

As used herein, the term “test sample” or “sample” generally refers to a biological material being tested for and/or suspected of containing an analyte of interest, such as a natriuretic peptide or natriuretic peptide fragment. The test sample may be derived from any biological source, such as, a physiological fluid, including, but not limited to, whole blood, serum, plasma, interstitial fluid, saliva, ocular lens fluid, cerebral spinal fluid, sweat, urine, milk, ascites fluid, mucous, nasal fluid, sputum, synovial fluid, peritoneal fluid, vaginal fluid, menses, amniotic fluid, semen and so forth. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release the analyte. Preferred test samples include urine, blood, serum and plasma.

### B. Methods for Detecting Autoantibodies Reactive to a Natriuretic Peptide or a Natriuretic Peptide Fragment in a Test Sample

In one embodiment, the present disclosure relates to methods for detecting or quantitating one or more autoantibodies that are reactive to a natriuretic peptide or natriuretic peptide fragment in a test sample. As discussed previously herein, the presence of autoantibodies to natriuretic peptides or natriuretic peptide fragments in a test sample can contribute to the generation of false negative results obtained in an assay. Therefore, the methods of the present disclosure allow one to learn prior to performing an assay whether or not a test sample might contain autoantibodies to a natriuretic peptide or natriuretic peptide fragment that might contribute to the generation of a false negative. Alternatively, the methods of the present disclosure provide one with a means necessary to confirm or question the correctness or reliability of a natriuretic peptide or natriuretic peptide fragment assay result.

The methods can be employed to detect or quantitate “one or more autoantibodies”, with “one or more” referring to types or populations. When employed to detect or quantitate more than one autoantibody, the autoantibodies detected are either directed against the same or different natriuretic peptides. In other words, with more than one autoantibody, the autoantibodies being detected are different from each other, either by being directed against a different natriuretic peptide, or by being a different form of antibody directed against the same natriuretic peptide (e.g., different region of the natriuretic peptide or peptide fragment).

The assay or method of the present disclosure involves obtaining a test sample from a subject. A subject from which a test sample can be obtained is any vertebrate. Preferably, the vertebrate is a mammal, especially a human. Examples of mammals include, but are not limited to, dogs, cats, rabbits, mice, rats, goats, sheep, cows, pigs, horses, non-human primates and humans. The test sample can be obtained from the subject using routine techniques known to those skilled in the art. Preferably, the test sample contains one or more autoantibodies reactive with a natriuretic peptide or natriuretic peptide fragment, and optionally the test sample further contains a natriuretic peptide or natriuretic peptide fragment.

The assay or method of the present disclosure can be performed in a homogeneous or heterogeneous format. It will be recognized by those skilled in the art that an essential difference between the two formats exists. For example, homogeneous formats lack one or more steps to separate the immunocomplex between and analyte of interest and a specific binding partner from the uncomplexed members. Further, homogeneous assays employ detectable labels. One or more characteristics of the signal generated from the detectable label are modulated by the formation of the immunocomplex. Such characteristics may include, but are not limited to, wavelength, intensity, duration or anisotropy. Examples of such homogeneous assays that can be used include, but are not limited to, fluorescence polarization immunoassay (FPIA), enzyme multiplied immunoassay technique (EMIT), bioluminescence resonance energy transfer (BRET), homogeneous chemiluminescent assay, etc. In a homogeneous format, after the test sample is obtained from a subject, a first mixture is prepared. The mixture contains the test sample being assessed for autoantibodies to a natriuretic peptide or natriuretic peptide fragment, and a first specific binding partner that is labeled with a detectable label. The first specific binding partner is a natriuretic peptide or a natriuretic peptide fragment. In terms of the detectable label, any detectable label known in the art can be used. For example, in FPIA, the detectable label can be a fluorescence label (See, for example U.S. Pat. Nos. 5,593,896, 5,573,904, 5,496,925, 5,359,093, and 5,352,803, incorporated herein by reference in their entirety). As further example, in a homogeneous chemiluminescent assay such as that described in Adamczyk, M., Chen, Y.-Y., Johnson, D. D., Mattingly, P. G., Moore, J. A., Pan, Y. and Reddy, R. E., Chemiluminescent Acridinium-9-carboxamide Boronic Acid Probes: Application to a Homogeneous Glycated Hemoglobin Assay, Bioorg. Med. Chem. Lett. 16:1324-1328 (2006); Adamczyk, M., Fino, J. R., Mattingly, P. G., Moore, J. A. and Pan, Y., Chemiluminescence quenching of pteroic acid-N-sulfonyl-acridinium-9-carboxamide conjugates by folate binding protein. Bioorg. Med. Chem. Lett. 14:2313-2317 (2004); Adamczyk, M., Johnson, D. D., Mattingly, P. G., Moore, J. A. and Pan, Y., Intrinsic factor-mediated modulation of cyanocobalamin-N-sulfonyl-acridinium-9-carboxamide chemiluminescence. Biorg. Med. Chem. Lett. 14:3917-3921 (2004); and Adamczyk, M., Mattingly, P. G., Moore, J. A. and Pan, Y. Regiodependent luminescence quenching of biotinylated N-sulfonyl-acridinium-9-carboxamides by avidin. Org. Lett., 5:3779-3782 (2003), the detectable label is an acridinium compound. Preferably, the acridinium compound is an acridinium-9-carboxamide. Specifically, the acridinium-9-carboxamide has a structure according to formula I:

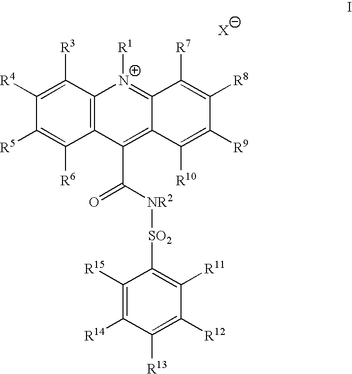

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl;

and further wherein any of the alkyl, alkenyl, alkynyl, aryl or aralkyl may contain one or more heteroatoms; and

optionally, if present,

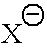

is an anion.

Methods for preparing acridinium 9-carboxamides are described in Mattingly, P. G. J. Biolumin. Chemilumin., 6, 107-14; (1991); Adamczyk, M.; Chen, Y.-Y., Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524 and 5,783,699 (each incorporated herein by reference in their entireties for their teachings regarding same).

Alternatively, the acridinium compound can be an acridinium-9-carboxylate aryl ester; the acridinium-9-carboxylate aryl ester can have a structure according to formula II:

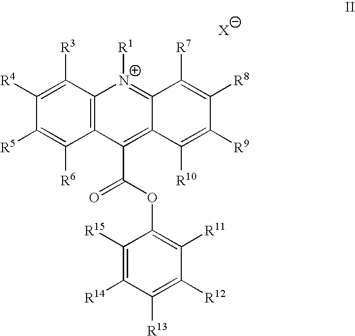

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

Examples of acridinium-9-carboxylate aryl esters having the above formula II that can be used in the present disclosure include, but are not limited to, 10-methyl-9-(phenoxycarbonyl)acridinium fluorosulfonate (available from Cayman Chemical, Ann Arbor, Mich.). Methods for preparing acridinium 9-carboxylate aryl esters are described in McCapra, F., et al., Photochem. Photobiol., 4, 1111-21 (1965); Razavi, Z et al., Luminescence, 15:245-249 (2000); Razavi, Z et al., Luminescence, 15:239-244 (2000); and U.S. Pat. No. 5,241,070 (each incorporated herein by reference in their entireties for their teachings regarding same).

The order in which the test sample and first specific binding partner labeled with the detectable label are added to form the mixture is not critical. After the first specific binding partner labeled with a detectable label and the test sample are added to form the first mixture, first specific binding partner-autoantibody complexes form.

In one embodiment of the present disclosure, hydrogen peroxide is generated in situ in the mixture or provided or supplied to the mixture before the addition of the above-described acridinium compound (specifically, the first specific binding partner labeled with the acridinium compound). In a second embodiment of the present disclosure, the hydrogen peroxide is generated in situ in the mixture or provided or supplied to the mixture simultaneously with the above-described acridinium compound (specifically, the first specific binding partner labeled with the acridinium compound). In a third embodiment, hydrogen peroxide is generated in situ or provided or supplied to the mixture after the above-described acridinium compound (specifically, the first specific binding partner labeled with the acridinium compound) is added to the test sample.

As mentioned above, hydrogen peroxide can be generated in situ in the mixture. Hydrogen peroxide can be generated in situ in a number of ways. For example, a hydrogen peroxide generating enzyme can be added to the first mixture. Specifically, one or more hydrogen peroxide generating enzymes can be added to the mixture in an amount sufficient to allow for the generation of hydrogen peroxide in situ in the mixture. The amount of one or more of the above enzymes to be added to the mixture can be readily determined by one skilled in the art.

Hydrogen peroxide can also be generated electrochemically in situ as shown in Agladze, G. R.; Tsurtsumia, G. S.; Jung, B. I.; Kim, J. S.; Gorelishvili, G. J. Applied Electrochem., 37, 375-383 (2007); Qiang, Z.; Chang, J.-H.; Huang, C.-P. Water Research, 36, 85-94 (2002), for example. Hydrogen peroxide can also be generated photochemically in situ, e.g. Draper, W. M.; Crosby, D. G. Archives of Environmental Contamination and Toxicology, 12, 121-126 (1983).

Alternatively, a source of hydrogen peroxide can be supplied to or provided in the mixture. For example, the source of the hydrogen peroxide can be one or more buffers or other solutions that are known to contain hydrogen peroxide. Such buffers or other solutions are simply added to the mixture. Alternatively, another source of hydrogen peroxide can simply be a solution containing hydrogen peroxide.

As demonstrated by the above, the timing and order in which the acridinium compound (specifically, the first specific binding partner labeled with the acridinium compound) and the hydrogen peroxide provided in or supplied to or generated in situ in the mixture is not critical provided that they are added, provided, supplied or generated in situ prior to the addition of at least one basic solution, which will be discussed in more detail below.

After the addition of the acridinium compound (specifically, the first specific binding partner labeled with the acridinium compound) and the hydrogen peroxide to the mixture, at least one basic solution is added to the mixture in order to generate a detectable signal, namely, a chemiluminescent signal. The basic solution is a solution that contains at least one base and that has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate. The amount of basic solution added to the mixture depends on the concentration of the basic solution used in the assay. Based on the concentration of the basic solution used, one skilled in the art could easily determine the amount of basic solution to be used in the method. Chemiluminescent signals generated can be detected using routine techniques known to those skilled in the art.

After the addition of the basic solution, a detectable signal from the detectable label is generated or emitted and then measured. Methods for generating signals from detectable labels and measuring the resulting signal generated are well known to those skilled in the art. For example, a chemiluminescent signal can be generated after the addition of a basic solution. The amount of the autoantibodies in the test sample can be quantified based on the intensity of the signal generated. Specifically, the amount of autoantibodies present can be quantified based on comparing the amount of light generated to a standard curve for autoantibodies to a natriuretic peptide or natriuretic peptide fragment or by comparison to a reference standard. The standard curve can be generated using serial dilutions or solutions of the autoantibodies to a natriuretic peptide or natriuretic peptide fragment of known concentration, by mass spectroscopy, gravimetrically and by other techniques known in the art.

In a heterogeneous format, after the test sample is obtained from a subject, a first mixture is prepared. The mixture contains the test sample being assessed for autoantibodies to a natriuretic peptide or natriuretic peptide fragment and a first specific binding partner, wherein the first specific binding partner and any autoantibodies contained in the test sample form a first specific binding partner-autoantibody complex. Preferably, the first specific binding partner is a natriuretic peptide or natriuretic peptide fragment. The order in which the test sample and first specific binding partner are added to form the mixture is not critical. Preferably, the first specific binding partner is immobilized on a solid phase. The solid phase used in the immunoassay (for the first specific binding partner and optionally, the second specific binding partner) can be any solid phase known in the art, such as, but not limited to, a magnetic particle, bead, test tube, microtiter plate, cuvette, membrane, a scaffolding molecule, film, filter paper, disc and chip.

After the mixture containing the first specific binding partner-autoantibody complex is formed, any unbound autoantibodies are removed from said complex using any technique known in the art, such as washing.

After any unbound autoantibodies are removed, a second specific binding partner is added to the mixture to form a first specific binding partner-autoantibody-second specific binding partner complex. The second specific binding partner is preferably an anti-human antibody. Moreover, also preferably, the second specific binding partner is labeled with or contains a detectable label. In terms of the detectable label, any detectable label known in the art can be used. For example, the detectable label can be a radioactive label (such as, e.g., ³H, ¹²⁵I, ³⁵S, ¹⁴C, ³²P, and ³³P), an enzymatic label (such as, e.g., horseradish peroxidase, alkaline peroxidase, glucose 6-phosphate dehydrogenase, and the like), a chemiluminescent label (such as, e.g., acridinium esters, thioesters, or sulfonamides; luminol, isoluminol, phenanthridinium esters, and the like), a fluorescence label (such as, e.g., fluorescein (e.g., 5-fluorescein, 6-carboxyfluorescein, 3′6-carboxyfluorescein, 5(6)-carboxyfluorescein, 6-hexachlorofluorescein, 6-tetrachlorofluorescein, fluorescein isothiocyanate, and the like)), rhodamine, phycobiliproteins, R-phycoerythrin, quantum dots (e.g., zinc sulfide-capped cadmium selenide), a thermometric label, or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997) and in Haugland, Handbook of Fluorescent Probes and Research Chemicals (1996), which is a combined handbook and catalogue published by Molecular Probes, Inc., Eugene, Oreg. Preferably, however, the detectable label is an acridinium compound that can be used in a chemiluminescent assay. Preferably, the acridinium compound is an acridinium-9-carboxamide. Specifically, the acridinium-9-carboxamide has a structure according to formula I:

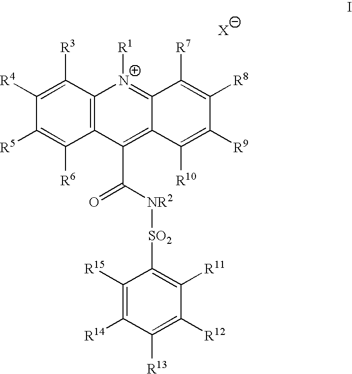

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl;

and further wherein any of the alkyl, alkenyl, alkynyl, aryl or aralkyl may contain one or more heteroatoms; and

optionally, if present,

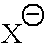

is an anion.

Methods for preparing acridinium 9-carboxamides are described in Mattingly, P. G. J. Biolumin. Chemilumin., 6, 107-14; (1991); Adamczyk, M.; Chen, Y.-Y., Mattingly, P. G.; Pan, Y. J. Org. Chem., 63, 5636-5639 (1998); Adamczyk, M.; Chen, Y.-Y.; Mattingly, P. G.; Moore, J. A.; Shreder, K. Tetrahedron, 55, 10899-10914 (1999); Adamczyk, M.; Mattingly, P. G.; Moore, J. A.; Pan, Y. Org. Lett., 1, 779-781 (1999); Adamczyk, M.; Chen, Y.-Y.; Fishpaugh, J. R.; Mattingly, P. G.; Pan, Y.; Shreder, K.; Yu, Z. Bioconjugate Chem., 11, 714-724 (2000); Mattingly, P. G.; Adamczyk, M. In Luminescence Biotechnology: Instruments and Applications; Dyke, K. V. Ed.; CRC Press: Boca Raton, pp. 77-105 (2002); Adamczyk, M.; Mattingly, P. G.; Moore, J. A.; Pan, Y. Org. Lett., 5, 3779-3782 (2003); and U.S. Pat. Nos. 5,468,646, 5,543,524 and 5,783,699 (each incorporated herein by reference in their entireties for their teachings regarding same).

Alternatively, the acridinium compound can be an acridinium-9-carboxylate aryl ester; the acridinium-9-carboxylate aryl ester can have a structure according to formula II:

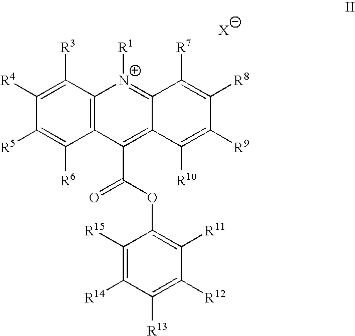

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

Examples of acridinium-9-carboxylate aryl esters having the above formula II that can be used in the present disclosure include, but are not limited to, 10-methyl-9-(phenoxycarbonyl)acridinium fluorosulfonate (available from Cayman Chemical, Ann Arbor, Mich.). Methods for preparing acridinium 9-carboxylate aryl esters are described in McCapra, F., et al., Photochem. Photobiol., 4, 1111-21 (1965); Razavi, Z et al., Luminescence, 15:245-249 (2000); Razavi, Z et al., Luminescence, 15:239-244 (2000); and U.S. Pat. No. 5,241,070 (each incorporated herein by reference in their entireties for their teachings regarding same).

After the addition of the second specific binding partner and after the formation of the first specific binding partner-autoantibody-second specific binding complex, any unbound second specific binding partner (whether labeled or unlabeled) is removed from said complex using any technique known in the art, such as washing.

In one embodiment of the present disclosure, hydrogen peroxide is generated in situ in the mixture or provided or supplied to the mixture before the addition of the above-described acridinium compound (specifically, the second specific binding partner labeled with the acridinium compound). In a second embodiment of the present disclosure, the hydrogen peroxide is generated in situ in the mixture or provided or supplied to the mixture simultaneously with the above-described acridinium compound (specifically, the second specific binding partner labeled with the acridinium compound). In a third embodiment, hydrogen peroxide is generated in situ or provided or supplied to the mixture after the above-described acridinium compound (specifically, the second specific binding partner labeled with the acridinium compound) is added to the test sample.

As mentioned above, hydrogen peroxide can be generated in situ in the mixture. Hydrogen peroxide can be generated in situ in a number of ways. For example, a hydrogen peroxide generating enzyme can be added to the first mixture. Specifically, one or more hydrogen peroxide generating enzymes can be added to the mixture in an amount sufficient to allow for the generation of hydrogen peroxide in situ in the mixture. The amount of one or more of the above enzymes to be added to the mixture can be readily determined by one skilled in the art.

Hydrogen peroxide can also be generated electrochemically in situ as shown in Agladze, G. R.; Tsurtsumia, G. S.; Jung, B. I.; Kim, J. S.; Gorelishvili, G. J. Applied Electrochem., 37, 375-383 (2007); Qiang, Z.; Chang, J.-H.; Huang, C.-P. Water Research, 36, 85-94 (2002), for example. Hydrogen peroxide can also be generated photochemically in situ, e.g. Draper, W. M.; Crosby, D. G. Archives of Environmental Contamination and Toxicology, 12, 121-126 (1983).

Alternatively, a source of hydrogen peroxide can be supplied to or provided in the mixture. For example, the source of the hydrogen peroxide can be one or more buffers or other solutions that are known to contain hydrogen peroxide. Such buffers or other solutions are simply added to the mixture. Alternatively, another source of hydrogen peroxide can simply be a solution containing hydrogen peroxide.

As demonstrated by the above, the timing and order in which the acridinium compound (specifically, the second specific binding partner labeled with the acridinium compound) and the hydrogen peroxide provided in or supplied to or generated in situ in the mixture is not critical provided that they are added, provided, supplied or generated in situ prior to the addition of at least one basic solution, which will be discussed in more detail below.

After the addition of the acridinium compound (specifically, the second specific binding partner labeled with the acridinium compound) and the hydrogen peroxide to the mixture, at least one basic solution is added to the mixture in order to generate a detectable signal, namely, a chemiluminescent signal. The basic solution is a solution that contains at least one base and that has a pH greater than or equal to 10, preferably, greater than or equal to 12. Examples of basic solutions include, but are not limited to, sodium hydroxide, potassium hydroxide, calcium hydroxide, ammonium hydroxide, magnesium hydroxide, sodium carbonate, sodium bicarbonate, calcium hydroxide, calcium carbonate and calcium bicarbonate. The amount of basic solution added to the mixture depends on the concentration of the basic solution used in the assay. Based on the concentration of the basic solution used, one skilled in the art could easily determine the amount of basic solution to be used in the method. Chemiluminescent signals generated can be detected using routine techniques known to those skilled in the art.

After any unbound second specific binding partner labeled with a detectable label is removed, a detectable signal from the detectable label is generated or emitted and then measured. Methods for generating signals from detectable labels and measuring the resulting signal generated are well known to those skilled in the art. For example, a chemiluminescent signal can be generated after the addition of a basic solution. The amount of the autoantibodies in the test sample can be quantified based on the intensity of the signal generated. Specifically, the amount of autoantibodies contained in a test sample is proportional to the intensity of the signal generated. Specifically, the amount of autoantibodies present can be quantified based on comparing the amount of light generated to a standard curve for autoantibodies to a natriuretic peptide or natriuretic peptide fragment or by comparison to a reference standard. The standard curve can be generated using serial dilutions or solutions of the autoantibodies to a natriuretic peptide or natriuretic peptide fragment of known concentration, by mass spectroscopy, gravimetrically and by other techniques known in the art.

The methods described herein can be used for determining the reliability of a result obtained from an assay (e.g., namely, an assay that was previously performed) for detecting or quantifying the amount of human natriuretic peptide in a test sample obtained from a subject. Specifically, such a method involves obtaining a test sample from a subject. Preferably, the test sample is obtained from the same subject for whom the assay was previously determined to detect or quantify the amount of human natriuretic peptide in the sample. Thus, this test sample would be the second or subsequent test sample obtained from the subject. After the second test sample is obtained from the subject, the concentration of amount of one or more human natriuretic peptide autoantibodies reactive with a human natriuretic peptide is determined using any of the assays described herein (e.g., using the methods described in this Section B), or any alternate assay for an autoantibody such as is known in the art, and including other than an immunoassay. If the concentration or amount of the one or more autoantibodies reactive with human natriuretic peptide in the second test sample is elevated compared to a predetermined level, then the result obtained from the previously performed assay is determined not to be reliable. However, if the concentration or amount of the one or more autoantibodies reactive with human natriuretic peptide in the second test sample is lower or the same as a predetermined level, then the result obtained from the previously performed assay is determined to be reliable.

As is apparent from the disclosure herein, generally a predetermined level can be employed as a benchmark against which to assess results obtained upon assaying a test sample for one or more autoantibodies reactive with a natriuretic peptide or natriuretic peptide fragment. Generally, in making such a comparison, the predetermined level is obtained by running a particular assay a sufficient number of times and under appropriate conditions such that a linkage or association of analyte (e.g., autoantibody) levels, concentrations or amounts with a particular endpoint of a disease, disorder or condition (e.g., cardiovascular disease), or with particular clinical indicia can be made. Typically, the predetermined level is obtained with assays of reference subjects (or populations of subjects) as described herein.

With respect to one or more autoantibodies reactive with a natriuretic peptide or natriuretic peptide fragment, it is envisioned that such autoantibodies can be directed against a variety of in vivo targets associated with the cardiovascular system or other major organ systems in which natriuretic peptide has any function or impact (e.g., central nervous system and/or respiratory system), and accordingly, that a particular autoantibody may either or increase or decrease with respect to a predetermined level based on the role or function of the antigen against which the autoantibody is directed.

In particular, with respect to a predetermined level as employed for monitoring disease progression and/or treatment, the concentration or amount of an autoantibody reactive with a natriuretic peptide or natriuretic peptide fragment may be either “unchanged,” “favorable” (or “favorably altered”), or “unfavorable” (or “unfavorably altered”). Generally, because autoantibodies as described herein appear to be associated with a natriuretic peptide-specific cardiophysiopathology and are elevated in a low proportion (e.g., less than about five %, especially from about 0.5% to about 5%) of the so-called normal population and are elevated in a higher proportion (less than about fifteen %, especially from about ten to about fifteen %) of the population testing positive for the presence of human natriuretic peptide (e.g., BNP), it is likely in most cases that “unfavorable” (“unfavorably altered”) corresponds to an increase or elevation in autoantibody amount or concentration, and “favorable” (“favorably altered”) corresponds to an decrease or reduction in autoantibody amount or concentration, in each case relative to a predetermined level or to a prior measured value.

As used herein, the term “elevated” or “increased” refers to a concentration or amount in a test sample that is higher than a typical or normal level or range (e.g., predetermined level), or is higher that another reference level or range (e.g., earlier or baseline sample). The term “lowered” or “reduced” refers to a concentration or amount in a test sample that is higher than a typical or normal level or range (e.g., predetermined level), or is higher that another reference level or range (e.g., earlier or baseline sample). The term “altered” refers to a concentration or amount in a sample that is altered (increased or decreased) over a typical or normal level or range (e.g., predetermined level), or over another reference level or range (e.g., earlier or baseline sample).

The typical or normal level or range for natriuretic peptide antigens and autoantibodies reactive therewith is defined in accordance with standard practice. Because the levels of autoantibodies in some instances will be very low, a so-called altered level or alteration can be considered to have occurred when there is any net change as compared to the typical or normal level or range, or reference level or range that cannot be explained by experimental error or sample variation. Thus, the level measured in a particular sample will be compared with the level or range of levels determined in similar samples from a so-called normal subject. In this context, a “normal subject” is an individual with no detectable cardiovascular pathology, and a “normal” (sometimes termed “control”) patient or population is/are one(s) that exhibits no detectable cardiovascular pathology. Furthermore, given that one or more autoantibodies against a natriuretic peptide are not routinely found at high levels in the majority of the human population, a “normal subject” can be considered an individual with no substantial detectable increased or elevated concentration or amount of analyte, and a “normal” (sometimes termed “control”) patient or population is/are one(s) that exhibits no substantial detectable increased or elevated concentration or amount of analyte. An “apparently normal subject” is one in which autoantibodies have not been or are being assessed. The level of an analyte is said to be “elevated” where the analyte is normally undetectable (e.g., the normal level is zero, or within a range of from about 25 to about 75 percentiles of normal populations), but is detected in a test sample, as well as where the analyte is present in the test sample at a higher than normal level.

As previously mentioned, it is believed that one or more autoantibodies reactive with a natriuretic peptide or natriuretic peptide fragment as described herein can be directed against a variety of in vivo targets associated with the cardiovascular system or other major organ systems in which natriuretic peptide has any function or impact (e.g., central nervous system and/or respiratory system). Thus, inter alia, the disclosure provides a method of screening for a subject having, or at risk of having cardiovascular disease, including but not limited to myocarditis, ischemic heart disease, or cardiomyopathy. In variations of these embodiments the cardiomyopathy is not dilated cardiomyopathy. Thus, for example, the method can be employed to screen for subjects having, or at risk of having, hypertrophic cardiomyopathy and/or restrictive cardiomyopathy.

Any of the test methods as described herein can be performed in conjunction with one or more other tests, including but not limited to physical examination, and/or the taking of a medical history to allow a differential diagnosis of, e.g., myocarditis, ischemic heart disease, or hypertrophic or restrictive cardiomyopathy. The various tests and parameters employed in diagnosing these disorders are well known to those of skill in the art. Furthermore, any of the methods can be carried out on samples from asymptomatic subjects or subjects having one or more risk factors associated with, or symptoms of, cardiovascular disease (including cardiovascular autoimmune disease_. For example, the subject may have an autoimmune disease, high blood pressure, or may have close (e.g., first-degree) relative with a heritable cardiovascular autoimmune disease, such as hypertrophic cardiomyopathy, or with an autoimmune disease that may deleteriously impact cardiovascular function (e.g., diabetes, rheumatic heart disease, or lupus).

In particular embodiments, when a subject is determined to have an unfavorable level of one or more autoantibodies to a natriuretic peptide, the subject optionally is assessed for one or more additional indicators of cardiovascular disease such as myoglobin, CK-MB (creatine kinase muscle-brain), BNP (brain natriuretic peptide), CRP (C reactive protein), cardiac troponin I (cTnI), cardiac troponin T (cTnT), blood oxygen level, cardiac imaging, electrocardiography, and any others that are known in the art.

However, such testing can optionally be carried out even when there has been no prior detection of an unfavorable level of one or more autoantibodies to a natriuretic peptide. For example, even in the absence of detection of such one or more autoantibodies, any the methods of the present disclosure can also be accompanied by measurement of one or more markers associated with cardiovascular disease. Such markers include natriuretic peptide or fragment thereof (e.g., combination antigen/antibody assay, or independent tests), as well as (and not limited to) pregnancy-associated plasma protein A (PAPP-A), IL-8, IL-10, interleukin-18 (IL-18/IL-18b), ischemic modified albumin (IMA), ICAM-1 (intercellular cell adhesion molecule-1), VCAM-1 (vascular cell adhesion molecule-1), fatty acid binding protein (FABP), E-selectin, P-selectin, fibrinogen, serum amyloid A (SAA), MPO (myeloperoxidase), LpPLA2 (lipoprotein-associated phospholipase A2), GP-BB (glycogen phosphorylase isoenzyme BB), IL1RA, TAFI (thrombin activable fibrinolysis inhibitor), soluble fibrin, anti-oxLDL (antibodies against oxidized low density lipoprotein), MCP-1 (monocyte chemoattractant protein-1), procoagulant tissue factor (TF), MMP-9 (matrix metalloproteinase 9), Ang-2 (angiopoietin-2), bFGF (basic fibroblast growth factor), VLDL (very low density lipoprotein), and PAI-1 (plasminogen activator inhibitor-1), among others.

Accordingly, the methods described herein can also be used to determine whether or not a subject has or is at risk of developing a cardiovascular disease. Specifically, such a method can comprise the steps of:

(a) determining the concentration or amount in a test sample from a subject of one or more autoantibodies reactive with human natriuretic peptide (e.g., using the methods described herein, or methods known in the art); and

(b) comparing the concentration or amount of the one or more autoantibodies reactive with human natriuretic peptide determined in step (a) with a predetermined level, wherein if the concentration or amount of the one or more autoantibodies reactive with human natriuretic peptide determined in step (a) is favorable with respect to a predetermined level, then the subject is determined not to have or be at risk for a cardiovascular disease. However, if the concentration or amount of the one or more autoantibodies reactive with human natriuretic peptide determined in step (a) is unfavorable with respect to the predetermined level then the subject is determined to have or be at risk for a cardiovascular disease.

Additionally, provided herein is method of monitoring the progression of disease in a subject. Optimally the method comprising the steps of:

(a) determining the concentration or amount in a first test sample from a subject of one or more autoantibodies reactive with human natriuretic peptide;

(b) determining the concentration or amount in a second or subsequent test sample from the subject of one or more autoantibodies reactive with human natriuretic peptide; and

(c) comparing the concentration or amount of one or more autoantibodies reactive with human natriuretic peptide determined in step (b) with the concentration or amount of one or more autoantibodies reactive with human natriuretic peptide determined in step (a), wherein if the concentration or amount determined in step (b) is unchanged or is unfavorable when compared to the concentration or amount of one or more autoantibodies reactive with human natriuretic peptide determined in step (a), then the disease in the subject is determined to have continued, progressed or worsened. By comparison, if the concentration or amount of one or more autoantibodies reactive with human natriuretic peptide determined in step (b) is favorable when compared to the concentration or amount of one or more autoantibodies reactive with human natriuretic peptide determined in step (a), then the disease in the subject is determined to have discontinued, regressed or improved.

Optionally the method further comprises comparing the concentration or amount of one or more autoantibodies reactive with human natriuretic peptide determined in step (b) or step (d) with a predetermined level. Further optionally the method comprises treating the subject with one or more pharmaceutical compositions for a period of time if the comparison shows that the concentration or amount of one or more autoantibodies reactive with human natriuretic peptide determined in step (b) or step (d) is unfavorably altered with respect to the predetermined level.

Still further, the methods of the present disclosure can be used to monitor treatment in a subject receiving treatment with one or more pharmaceutical compositions. Specifically, such methods involve providing a first test sample from a subject before the subject has been administered one or more pharmaceutical compositions. Next, the concentration or amount in a first test sample from a subject of one or more autoantibodies reactive with human natriuretic peptide is determined (e.g., using the methods described herein or as known in the art). After the concentration or amount of one or more autoantibodies reactive with human natriuretic peptide is determined is determined, optionally the concentration or amount of one or more autoantibodies reactive with human natriuretic peptide is determined is then compared with a predetermined level. If the concentration or amount of the one or more autoantibodies reactive with human natriuretic peptide determined in the first test sample is lower than the predetermined level, then the subject is not treated with one or more pharmaceutical compositions. However, if the concentration or amount of the one or more autoantibodies reactive with human natriuretic peptide determined in the first test sample is higher than the predetermined level, then the subject is treated with one or more pharmaceutical compositions for a period of time. The period of time that the subject is treated with the one or more pharmaceutical compositions can be determined by one skilled in the art (for example, the period of time can be from about seven (7) days to about two years, preferably from about fourteen (14) days to about one (1) year).

During the course of treatment with the one or more pharmaceutical compositions, second and subsequent test samples are then obtained from the subject. The number of test samples and the time in which said test samples are obtained from the subject are not critical. For example, a second test sample could be obtained seven (7) days after the subject is first administered the one or more pharmaceutical compositions, a third test sample could be obtained two (2) weeks after the subject is first administered the one or more pharmaceutical compositions, a fourth test sample could be obtained three (3) weeks after the subject is first administered the one or more pharmaceutical compositions, a fifth test sample could be obtained four (4) weeks after the subject is first administered the one or more pharmaceutical compositions, etc.

After each second or subsequent test sample is obtained from the subject, the concentration or amount of one or more autoantibodies reactive with human natriuretic peptide is determined in the second or subsequent test sample is determined (e.g., using the methods described herein or as known in the art). The concentration or amount of one or more autoantibodies reactive with human natriuretic peptide is determined determined in each of these second or subsequent test samples is then compared with the concentration or amount of one or more autoantibodies reactive with human natriuretic peptide is determined determined in the first test sample (e.g., the test sample that was originally optionally compared to the predetermined level). If the concentration or amount of one or more autoantibodies reactive with human natriuretic peptide determined in step (c) is favorable when compared to the concentration or amount of one or more autoantibodies reactive with human natriuretic peptide determined in step (a), then the disease in the subject is determined to have discontinued, regressed or improved, and the subject should continue to be administered the one or pharmaceutical compositions of step (b). However, if the concentration or amount determined in step (c) is unchanged or is unfavorable when compared to the concentration or amount of one or more autoantibodies reactive with human natriuretic peptide determined in step (a), then the disease in the subject is determined to have continued, progressed or worsened, and the subject should be treated with a higher concentration of the one or more pharmaceutical compositions administered to the subject in step (b) or the subject should be treated with one or more pharmaceutical compositions that are different then the one or more pharmaceutical compositions administered to the subject in step (b). Specifically, the subject can be treated with one or more pharmaceutical compositions that are different then the one or more pharmaceutical compositions that the subject had previously received to decrease or lower said subject's human natriuretic peptide autoantibodies levels.

Generally, for assays in which repeat testing may be done (e.g., monitoring disease progression and/or response to treatment), a second or subsequent test sample is obtained at a period in time after the first test sample has been obtained from the subject. Specifically, second test sample from the subject can be obtained minutes, hours, days, weeks or years after the first test sample has been obtained from the subject. For example, the second test sample can be obtained from the subject at a time period of about 1 minute, about 5 minutes, about 10 minutes, about 15 minutes, about 30 minutes, about 45 minutes, about 60 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years after the first test sample from the subject is obtained. Where used to monitor disease progression, the above assay can be used to monitor the progression of disease in subjects suffering from acute conditions. Acute conditions, also known as critical care conditions, refer to acute, life threatening diseases or other critical medical conditions involving the cardiovascular system (including, but not limited to, sepsis, as well as systemic inflammatory response syndromes), central nervous stem and/or respiratory system. Typically, critical care conditions refer to those conditions requiring acute medical intervention in a hospital based setting (including, but not limited to, the emergency room, intensive care unit, trauma center or other emergent care setting) or administration by a paramedic or other field-based medical personnel. For critical care conditions, repeat monitoring is generally done within a shorter time frame, namely, minutes, hours or days (e.g., about 1 minute, about 5 minutes, about 10 minutes, about 15 minutes, about 30 minutes, about 45 minutes, about 60 minutes, about 2 hours, about 3 hours, about 4 hours, 4about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days or about 7 days), and the initial assay likewise is generally done within a shorter timeframe, e.g., about minutes, hours or days of the onset of the disease or condition.

The assays also can be used to monitor the progression of disease in subjects suffering from chronic, or non-acute conditions. Non-critical care or, non-acute conditions, refers to conditions other than acute, life threatening disease or other critical medical conditions involving the cardiovascular system, central nervous system and/or respiratory system. Typically, non-acute conditions include those of longer-term or chronic duration, and include, e.g., ophthalmic conditions and cancer. For non-acute conditions, repeat monitoring generally is done with a longer timeframe, e.g., hours, days, weeks, months or years (e.g., about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 21 weeks, about 22 weeks, about 23 weeks, about 24 weeks, about 25 weeks, about 26 weeks, about 27 weeks, about 28 weeks, about 29 weeks, about 30 weeks, about 31 weeks, about 32 weeks, about 33 weeks, about 34 weeks, about 35 weeks, about 36 weeks, about 37 weeks, about 38 weeks, about 39 weeks, about 40 weeks, about 41 weeks, about 42 weeks, about 43 weeks, about 44 weeks, about 45 weeks, about 46 weeks, about 47 weeks, about 48 weeks, about 49 weeks, about 50 weeks, about 51 weeks, about 52 weeks, about 1.5 years, about 2 years, about 2.5 years, about 3.0 years, about 3.5 years, about 4.0 years, about 4.5 years, about 5.0 years, about 5.5. years, about 6.0 years, about 6.5 years, about 7.0 years, about 7.5 years, about 8.0 years, about 8.5 years, about 9.0 years, about 9.5 years or about 10.0 years), and the initial assay likewise generally is done within a longer time frame, e.g., about hours, days, months or years of the onset of the disease or condition.

Furthermore, the above assays can be performed using a first test sample obtained from a subject where the first test sample is whole blood, serum or plasma. The above assays can then be repeated using a second test sample obtained from the subject where the second test sample is something other than whole blood, serum or plasma (e.g., urine). The results obtained from the assays using the first test sample and the second test sample are compared. The comparison can be used to assess the status of a disease or condition in the subject.

Moreover, the present disclosure also relates to methods of determining whether a subject predisposed to or suffering from a disease (e.g., cardiovascular disease) will benefit from treatment. In particular, the disclosure relates to natriuretic peptide companion diagnostic methods and products. Thus, the method of “monitoring the treatment of disease in a subject” as described herein further optimally also can encompass selecting or identifying candidates for therapy.

Thus, in particular embodiments, the disclosure also provides a method of determining whether a subject having, or at risk for, cardiovascular disease (e.g., cardiovascular autoimmune disease) is a candidate for therapy (e.g., immunosuppressive therapy or immunoabsorption therapy, or treatment with one or more pharmaceutical compositions). Generally, the subject is one who has experienced some symptom of cardiovascular disease (e.g., cardiovascular autoimmune disease) or who has actually been diagnosed as having, or being at risk for, cardiovascular disease (e.g., cardiovascular autoimmune disease), and/or who demonstrates an unfavorable concentration or amount of one or more autoantibodies reactive with a natriuretic peptide or fragment thereof, as described herein.

The method optionally comprises an assay as described herein, where analyte is assessed before and following treatment of a subject with one or more pharmaceutical compositions (e.g., particularly with a pharmaceutical related to a natriuretic peptide mechanism of action, e.g., BNP, nesiritide, or any combination thereof), with immunosuppressive therapy, or by immunoabsorption therapy, or where analyte is assessed following such treatment and concentration or amount of analyte is compared against a predetermined level. An unfavorable concentration of amount of analyte observed following treatment confirms that the subject will not benefit from receiving further or continued treatment; whereas a favorable concentration of amount of analyte observed following treatment confirms that the subject will benefit from receiving further or continued treatment. This confirmation assists with management of clinical studies, and provision of improved patient care.

### C. Isolated Human Natriuretic Peptide Autoantibodies

In another embodiment, the present disclosure relates to isolated human natriuretic peptide autoantibodies. The isolated human natriuretic peptide autoantibodies of the present disclosure can be pre-pro peptide precursor human ANP autoantibodies, pro peptide human ANP autoantibodies, N-terminal pro peptide human ANP autoantibodies, human ANP autoantibodies, pre-pro peptide precursor human BNP autoantibodies, pro peptide human BNP autoantibodies, N-terminal pro peptide human BNP autoantibodies, human BNP autoantibodies, human CNP autoantibodies, pro peptide human CNP autoantibodies or any combinations thereof. The isolated human natriuretic peptide autoantibodies can be IgG, IgA or IgM antibodies. Preferably, the isolated autoantibodies are IgG antibodies. In one embodiment, the isolated human natriuretic peptide autoantibodies are human prohBNP autoantibodies. In another embodiment, the isolated human natriuretic peptide autoantibodies are hBNP autoantibodies.

The isolated human natriuretic peptide autoantibodies of the present disclosure can be obtained using routine techniques known in the art. For example, the autoantibodies can be obtained by separating such autoantibodies from their environment, such as for example, from a mixture containing human natriuretic peptide autoantibodies. Such a mixture could be obtained from a subject that has been receiving treatment with a human natriuretic peptide (e.g., treatment with at least one of one pre-pro peptide precursor of human ANP, pro peptide of human ANP, N-terminal pro peptide of ANP, human ANP, pre-pro peptide precursor of human BNP, pro peptide of human BNP, N-terminal pro peptide of BNP, human BNP, human CNP, pro peptide of human CNP or any combinations thereof), human natriuretic peptide fragment (e.g., treatment with at least one of human A-type natriuretic peptide fragment, human B-type natriuretic peptide fragment, human C-type natriuretic peptide fragment or any combinations thereof) or human natriuretic peptide derivative. Alternatively, the mixture could be obtained from a subject with the clinical symptoms of congestive heart failure or an endogeneous concentration of a human natriuretic peptide, human natriuretic peptide fragment or human natriuretic peptide derivative that is higher than the clinically acceptable value for a normal population.

Mixtures containing such autoantibodies can be readily identified using the methods described above in Section B, and in the Examples. For instance, the Examples describe the identification of certain subject populations having elevated levels of one or more autoantibodies reactive with natriuretic peptide. Such a mixture can be obtained using routine techniques known in the art, for example, by obtaining a blood, plasma or serum sample from a subject.

As mentioned briefly above, the autoantibodies of the present disclosure can be isolated from their environment using routine immunoglobulin procedures such as, for example, salt fractionation (such as ammonium sulfate precipitation), immunoprecipitation, affinity capture on a solid phase, gel electrophoresis, dialysis, or chromatography (such as protein A-Sepharose chromatography, hydroxyapatite chromatography, affinity chromatography, anion-exchange chromatography, ion-exchange chromatography, immunoaffinity chromatography, size exclusion chromatography, reversed-phase chromatography, etc.).

In particular, the specific binding partners described herein and in the following Examples can be employed using standard techniques in the isolation of autoantibody and/or confirmation of identification of the isolated autoantibody, particularly an autoantibody reactive with human proBNP or hBNP.

Once such isolated human natriuretic peptide autoantibodies are obtained, the amino acid sequence of such antibodies can be determined using routine techniques known in the art, such as by use of automated Edman degradation. Alternatively, the amino acid sequence of such antibodies can be determined using mass spectrometry methods and techniques of as describe in Adamczyk, M., Gebler, J. C., Wu, J., and Yu, Z., “Complete sequencing of anti-vancomycin fab fragment by liquid chromatography-electrospray ion trap mass spectrometry with a combination of database searching and manual interpretation of the MS/MS spectra”. J Immunol Methods 260, 235-49 (2002); Adamczyk, M., Gebler, J. C., and Wu, J., “Sequencing of anti-thyroxine monoclonal antibody fab fragment by ion trap mass spectrometry”. Rapid Commun Mass Spectrom 14, 999-1007 (2000); Adamczyk, M., Gebler, J. C., and Wu, J., “Papain digestion of different mouse IgG subclasses as studied by electrospray mass spectrometry”. J Immunol Methods 237, 95-104 (2000); Adamczyk, M., Gebler, J. C., and Wu, J., “Profiling of polyclonal antibody light chains by liquid chromatography/electrospray ionization mass spectrometry”. Rapid Commun Mass Spectrom 14, 49-51 (2000); Adamczyk, M., Gebler, J. C., and Wu, J., “A simple method to identify cysteine residues by isotopic labeling and ion trap mass spectrometry”. Rapid Commun Mass Spectrom 13, 1813-7 (1999); and Adamczyk, M., Gebler, J. C., and Wu, J., “Charge derivatization of peptides to simplify their sequencing with an ion trap mass spectrometer”. Rapid Commun Mass Spectrom 13, 1413-22 (1999).

Once the amino acid sequence of the autoantibodies is determined the nucleic acid sequence of such autoantibodies can also be determined using routine techniques known in the art. The nucleic acid sequence thus obtained may be used to express the autoantibodies or autoantibody fragments in human or non-human cell lines, or non-human subjects as well known to those skilled in the art of antibody engineering.

Additionally, once the amino acid and nucleic acid sequence of the isolated human natriuretic peptide autoantibodies is obtained, the amino acid and nucleic acid sequences can be directly synthesized using various solid-phase techniques (See, Roberge J Y et al., Science 269:202-204 (1995)) and automated synthesis may be achieved, for example, using the ABI 43 1 A Peptide Synthesizer (Perkin Elmer) in accordance with the instructions provided by the manufacturer. Additionally, the amino acid sequences obtainable from the isolated autoantibodies described herein may be altered during direct synthesis and/or combined using chemical methods with a sequence from other subunits, or any part thereof, to produce variant sequences and hence variant human natriuretic peptide autoantibodies.

The human natriuretic peptide autoantibodies described herein can be used for a variety of different purposes. For example, these isolated autoantibodies can be used in screening methods to identify agents that are useful in inhibiting the binding of at least one human natriuretic peptide or natriuretic peptide fragment to at least one human anti-natriuretic peptide autoantibody. Specifically, such screening methods would involve preparing a mixture comprising an isolated human natriuretic peptide autoantibody. After such a mixture is prepared, the method would involve adding to the mixture, either simultaneously or sequentially, and in any order, at least one human natriuretic peptide or natriuretic peptide fragment and at least one agent to best tested (such as a pharmaceutical composition, etc.). The final step would involve determining whether the agent being tested inhibits the binding of the at least one human natriuretic peptide or natriuretic peptide fragment to the human natriuretic peptide autoantibody. It is contemplated that such a method could be partially or fully automated to allow for the screening of a large number of agents at one time. Agents determined to inhibit the binding of at least one human natriuretic peptide or natriuretic peptide fragment to the human natriuretic peptide autoantibody would be selected for further study use as a potential therapeutic agent in treating cardiovascular disease in a human. Additionally, the isolated autoantibodies described herein could also be employed in pharmaceutical compositions that can be used to treat cardiovascular disease. Such pharmaceutical compositions would contain the isolated autoantibodies described herein and one or more pharmaceutically acceptable excipients.

### D. Assay Kits for Detecting One or More Autoantibodies Reactive With a Natriuretic Peptide or a Natriuretic Peptide Fragment in a Test Sample

In another embodiment, the present disclosure relates to a test kit for detecting one or more autoantibodies that are reactive to a natriuretic peptide or natriuretic peptide fragment in a test sample. The kit can contain a first specific binding partner, wherein said first specific binding partner is a natriuretic peptide or natriuretic peptide fragment. Optionally, the kit can also contain a second specific binding partner wherein said second specific binding partner is an anti-human antibody. The kit can also contain at least one detectable label. The detectable label can be a separate component of the kit. Alternatively, the detectable label may be conjugated to the first or second specific binding partner and supplied in the kit in this form. Preferably, the detectable label is at least one acridinium compound. If the kit contains at least one acridinium compound, the acridinium compound may comprise at least one acridinium-9-carboxamide, at least one acridinium-9-carboxylate aryl ester or any combinations thereof. More specifically, the acridinium-9-carboxamide that can be used has the structure according to Formula I:

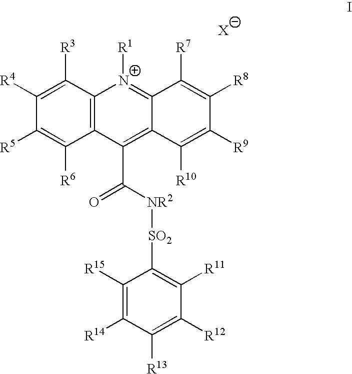

wherein R¹ and R² are each independently selected from the group consisting of: alkyl, alkenyl, alkynyl, aryl or aralkyl, sulfoalkyl, carboxyalkyl and oxoalkyl, and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

further wherein any of the alkyl, alkenyl, alkynyl, aryl or aralkyl may contain one or more heteroatoms; and

optionally, if present, X^{⊖} is an anion.

Additionally, the acridinium-9-carboxylate aryl ester that can be used has a structure according to formula II:

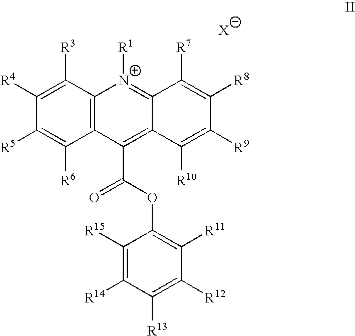

wherein R¹ is an alkyl, alkenyl, alkynyl, aryl or aralkyl, sulfoalkyl, carboxyalkyl and oxoalkyl; and

wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and

optionally, if present, X^{⊖} is an anion.

Additionally, the kit can also contain a means of generating hydrogen peroxide in situ in the test sample. A means for generating hydrogen peroxide in situ in the test sample can include adding at least one hydrogen peroxide generating enzyme. Alternatively, the kit can contain at least one source of hydrogen peroxide. The at least one source of hydrogen peroxide can be one or more buffers or other solutions that are known to contain hydrogen peroxide. Alternatively, the kit can contain a solution containing hydrogen peroxide.

Additionally, the kit can also contain at least one basic solution.

Additionally, the kit can also contain a solid phase. For example, the solid phase can be a magnetic particle, bead, test tube, microtiter plate, cuvette, membrane, a scaffolding molecule, film, filter paper, disc and chip.

Also, the kit can also contain one or more instructions for detecting and quantifying autoantibodies to a natriuretic peptide or natriuretic peptide fragment in a test sample. The kit can also contain instructions for generating a standard curve for the purposes of quantifying the autoantibodies or a reference standard for purposes of quantifying the autoantibodies in the test sample. Such instructions optionally can be in printed form or on CD, DVD, or other format of recorded media.

### E. Adaptations of the Methods and Kits of the Present Disclosure

The disclosure as described herein also can be adapted for use in a variety of automated and semi-automated systems (including those wherein the solid phase comprises a microparticle), as described, e.g., in U.S. Pat. Nos. 5,089,424 and 5,006,309, and as, e.g., commercially marketed by Abbott Laboratories (Abbott Park, Ill.) including but not limited to Abbott's ARCHITECT®, AxSYM, IMX, PRISM, and Quantum II instruments, as well as other platforms. Moreover, the disclosure optionally is adaptable for the Abbott Laboratories commercial Point of Care (I-STAT®) electrochemical immunoassay system for performing sandwich immunoassays. Immunosensors, and their methods of manufacture and operation in single-use test devices are described, for example in, U.S. Pat. No. 5,063,081, U.S. Patent Application 2003/0170881, U.S. Patent Application 2004/0018577, U.S. Patent Application 2005/0054078, and U.S. Patent Application 2006/0160164, which are incorporated in their entireties by reference for their teachings regarding same.

In particular, with regard to the adaptation of the present autoantibody assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized first specific binding partner (natriuretic peptide or natriuretic peptide fragment) are adhered to a polymer coating of patterned polyvinyl alcohol over the electrode. This chip is assembled into an I-STAT® cartridge with a fluidics format suitable for immunoassay. On a portion of the wall of the sample holding chamber of the cartridge there is a layer comprising the second natriuretic peptide specific binding partner labeled with alkaline phosphatase (or other label). Within the fluid pouch of the cartridge is an aqueous reagent that includes p-aminophenol phosphate.

In operation, a sample suspected of containing natriuretic peptide is added to the holding chamber of the natriuretic peptide test cartridge and the cartridge is inserted into the I-STAT® reader. After the second specific binding partner has dissolved into the sample, a pump element within the cartridge forces the sample into a conduit containing the chip. Here it is oscillated to promote formation of the sandwich between the first specific binding partner, natriuretic peptide and the labeled second specific binding partner. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of natriuretic peptide in the sample by means of an embedded algorithm and factory-determined calibration curve.

It further goes without saying that the methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), other salt, protein blockers, antimicrobial and detergent. An exemplary calibrator diluent is ARCHITECT® Human calibrator diluent employed in certain kits (Abbott Laboratories, Abbott Park, Ill.), which comprises a buffer containing MES, other salt, a protein blocker and an antimicrobial.

Furthermore, as previously mentioned, the methods and kits optionally are adapted for use on an automated or semi-automated system. Some of the differences between an automated or semi-automated system as compared to a non-automated system (e.g., ELISA) include the substrate to which the first specific binding partner (e.g., analyte antigen or capture antibody) is attached (which can impact sandwich formation and analyte reactivity), and the length and timing of the capture, detection and/or any optional wash steps. Whereas a non-automated format such as an ELISA may include a relatively longer incubation time with sample and capture reagent (e.g., about 2 hours) an automated or semi-automated format (e.g., ARCHITECT®) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT®) may have a relatively shorter incubation time (e.g., approximately 4 minutes for the ARCHITECT®).

By way of example, and not of limitation, examples of the present disclosures shall now be given.

### EXAMPLE 1

### Preparation and Characterization of Natriuretic Peptide Coated Microplates

Recombinant pro peptide of human BNP (BiosPacific, Emeryville, Calif., Catalog No. J10710359) was dissolved in phosphate buffer (0.2 M, pH 8.0) to give a solution of 2 μg/mL. Microplates (Costar®, Sigma-Aldrich, St. Louis, Mo., Catalog No. 3923) were coated with the pro peptide of human BNP solution (100 μL/well) for 2 hours at 38° C. with mixing at 28 rpm. The plates were drained, and the pro peptide of human BNP solution was replaced with a solution of heat-inactivated bovine serum albumin (BSA, 2% w/v in PBS, 300 μL/well). The plates were incubated at 38° C. for 1 hour with mixing at 28 rpm, and then drained. The plates were then washed three times with a solution of sucrose (2% w/v in PBS, 300 μL/well), drained, and dried under a stream of dry nitrogen.

A murine anti-hBNP antibody (Scios Inc., Mountain View, Calif., Mab 106.3, 100 ng/mL) labeled with an acridinium-9-carboxamide was added to a pro-peptide of human BNP coated microplate (100 μL/well) and compared to a murine anti-human antibody labeled with an acridinium-9-carboxamide (200 ng/mL) added to a pro-BNP coated microplate (100 μL/well). The hBNP reactive antibody conjugate gave 180-fold higher response than the anti-human IgG conjugate (See Table 2, below) which demonstrates the effectiveness of the coating procedure to form a solid-supported, immunoreactive pro-BNP surface.

TABLE 2
Reactivity of anti-hBNP antibodies with pro-BNP-coated microplates

| Conjugate | Average RLUmax | % CV |
|---|---|---|
| Anti-hBNP 106.3 | 954650 | 5 |
| Anti-human IgG | 5310 | 16 |

### EXAMPLE 2

### Analysis of Human Plasma for Endogenous Antibodies Reactive with Natriuretic Peptides

Samples: Frozen donor plasma from apparently healthy individuals (meaning no reported disease or symptoms of disease), cardiac troponin-I (cTnI) positive plasma samples, and hBNP positive plasma samples, were obtained from Abbott Laboratories (Abbott Park, Ill.) specimen bank and thawed at 2-8° C. prior to use.

Microplate preparation: Microplates were prepared according to the method in Example 1.

Chemiluminescent detection conjugate: A murine anti-human IgG (subtype IgG2b, kappa) was labeled with a chemiluminescent acridinium-9-carboxamide. This antibody recognized all human IgG subtypes while having no significant reactivity toward human IgM or IgA, or rabbit, sheep or goat IgG.

Assay protocol: The sample (10 μL) was diluted with AxSYM® Troponin-I ADV (Abbott Laboratories, Abbott Park, Ill.) Preincubation Diluent (90 μL) in the microplate well. After incubating at 37° C. for 2 hours, the plate was washed with ARCHITECT® Wash Buffer (3×, 350 μL). The murine anti-human IgG specific monoclonal-acridinium conjugate (100 μL) was then added and the plate incubated at 37° C. for 1 h, before a final wash with ARCHITECT® Wash Buffer (3×, 350 μL).

Chemiluminescent detection: The microplate was loaded into a Mithras microplate reader (Berthold Technologies Inc, Oak Ridge, Tenn.) equilibrated at 28° C. The chemiluminescence signal from each well was recorded for 2 seconds after the sequential addition of ARCHITECT® Pre-Trigger solution (100 μL) and ARCHITECT® Trigger solution (100 μL).

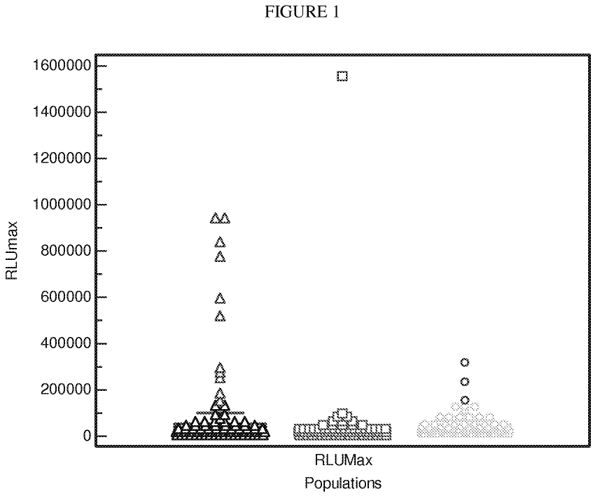

General statistics for the populations tested are shown in Table 3, below. The population data are presented in a distribution plot (See, FIG. 1). As shown, 3% (3/97) of the apparently healthy donor population; 1.2% (1/77) of the cardiac troponin-I positive population; and 11.4% (11/96) of the BNP positive population have very high levels of human proBNP-reactive autoantibodies i.e., RLUmax greater than the upper quartile plus 1.5 times the interquartile range.

Summary statistics are presented in Tables 3-6 below.

TABLE 3
Study Total Population Data (all Subgroups Combined)

| Variable | RLUmax |
|---|---|
| Sample size | 270 |
| Lowest value | 4390.0000 |
| Highest value | 1552860.0000 |
| Arithmetic mean | 62486.5926 |
| 95% CI for the mean | 44598.9554 to 80374.2298 |
| Median | 28610.0000 |
| 95% CI for the median | 25520.1920 to 32206.5793 |
| Variance | 22287267430.7256 |
| Standard deviation | 149289.2073 |
| Relative standard deviation | 2.3891 (238.91%) |
| Standard error of the mean | 9085.4518 |
| Coefficient of Skewness | 6.5345 (P < 0.0001) |
| Coefficient of Kurtosis | 49.8031 (P < 0.0001) |
| D'Agostino-Pearson test | Reject Normality (P < 0.0001) |
| for Normal distribution | |

| Percentiles | | 95% Confidence Interval |
|---|---|---|
| 2.5 | 9122.5000 | 6502.3740 to 10792.0295 |
| 25 | 19120.0000 | 17285.9590 to 21292.1263 |
| 75 | 46770.0000 | 41103.6674 to 55323.6752 |
| 90 | 86135.0000 | 67732.4508 to 131702.7572 |
| 97.5 | 469387.5000 | 154138.6714 to 943632.3615 |

TABLE 4
Subgroup Population hBNP-Positive Data

| | |
|---|---|
| Sample size | 96 |
| Lowest value | 7260.0000 |
| Highest value | 944340.0000 |
| Arithmetic mean | 90428.6458 |
| 95% CI for the mean | 52280.4338 to 128576.8579 |
| Median | 28610.0000 |
| 95% CI for the median | 22604.3845 to 36599.5445 |
| Variance | 35447756984.4627 |
| Standard deviation | 188275.7472 |
| Relative standard deviation | 2.0820 (208.20%) |
| Standard error of the mean | 19215.8130 |
| Coefficient of Skewness | 3.5371 (P < 0.0001) |
| Coefficient of Kurtosis | 12.1051 (P < 0.0001) |
| D'Agostino-Pearson test | Reject Normality (P < 0.0001) |
| for Normal distribution | |

| Percentiles | | 95% Confidence Interval |
|---|---|---|
| 2.5 | 10707.0000 | |
| 25 | 20355.0000 | 17294.4176 to 21993.8259 |
| 75 | 52530.0000 | 40792.2764 to 76756.0576 |
| 90 | 183821.0000 | 67773.7195 to 662404.1283 |
| 97.5 | 851501.0000 | |

TABLE 5
Subgroup Population cTnI-Positive Data

| | |
|---|---|
| Sample size | 77 |
| Lowest value | 4390.0000 |
| Highest value | 1552860.0000 |
| Arithmetic mean | 48171.5584 |
| 95% CI for the mean | 8503.8298 to 87839.2871 |
| Median | 24130.0000 |
| 95% CI for the median | 20123.5189 to 29668.3192 |
| Variance | 30544251789.6446 |
| Standard deviation | 174769.1386 |
| Relative standard deviation | 3.6281 (362.81%) |
| Standard error of the mean | 19916.7918 |
| Coefficient of Skewness | 8.6176 (P < 0.0001) |
| Coefficient of Kurtosis | 75.1148 (P < 0.0001) |
| D'Agostino-Pearson test | Reject Normality(P < 0.0001) |
| for Normal distribution | |

| Percentiles | | 95% Confidence Interval |
|---|---|---|
| 2.5 | 6085.7500 | |
| 25 | 14957.5000 | 12220.1948 to 19597.6255 |
| 75 | 36292.5000 | 29863.2375 to 44584.8208 |
| 90 | 56276.0000 | 38772.6618 to 87998.8865 |
| 97.5 | 94335.2500 | |

TABLE 6
Subgroup Population Normal Donor Data

| | |
|---|---|
| Sample size | 97 |
| Lowest value | 10100.0000 |
| Highest value | 316770.0000 |
| Arithmetic mean | 46196.0825 |
| 95% CI for the mean | 37232.1295 to 55160.0354 |
| Median | 32770.0000 |
| 95% CI for the median | 27793.5691 to 39892.2771 |
| Variance | 1978138576.1598 |
| Standard deviation | 44476.2698 |
| Relative standard deviation | 0.9628 (96.28%) |
| Standard error of the mean | 4515.8810 |
| Coefficient of Skewness | 3.5290 (P < 0.0001) |
| Coefficient of Kurtosis | 16.4805 (P < 0.0001) |
| D'Agostino-Pearson test | Reject Normality (P < 0.0001) |
| for Normal distribution | |

| Percentiles | | 95% Confidence Interval |
|---|---|---|
| 2.5 | 10918.5000 | |
| 25 | 21630.0000 | 18209.3643 to 26466.4464 |
| 75 | 54040.0000 | 42974.8751 to 69257.1686 |
| 90 | 85880.0000 | 62821.4107 to 132329.7869 |
| 97.5 | 159904.5000 | |

These results confirm that, surprising and unexpectedly, one or more autoantibodies reactive with human natriuretic peptide (e.g., human proBNP) are found in apparently healthy individuals. The further trend identified above suggests that the more or more autoantibodies are indicators of and potentially associated with cardiac physiopathology. Specifically, high levels of human proBNP-reactive autoantibodies are found at a low level in the apparently normal population (3% in an apparently normal donor population), are by comparison reduced in the cardiac troponin-I positive population (1.2%), and are by comparison increased in the BNP positive population (11.4%). Both cardiac troponin-I and BNP are approved biomarkers for cardiovascular disease. These results suggest that natriuretic peptide-reactive autoantibodies may be associated with cardiac pathologies. Of course, these unexpected and important findings have ramifications in terms of prognosis, disease monitoring and treatment.

One skilled in the art would readily appreciate that the present disclosure is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the disclosure. It will be readily apparent to one skilled in the art that varying substitutions and modifications may be made to the disclosure disclosed herein without departing from the scope and spirit of the disclosure.

All patents and publications mentioned in the specification are indicative of the levels of those skilled in the art to which the disclosure pertains. All patents and publications are herein incorporated by reference to the same extent as if each individual publication was specifically and individually indicated to be incorporated by reference.

The disclosure illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

What is claimed is:

1. A method for detecting one or more autoantibodies reactive with at least one natriuretic peptide or natriuretic peptide fragment in a test sample, the method comprising the steps of:
(a) preparing a mixture comprising a test sample being assessed for the presence of one or more autoantibodies to at least one natriuretic peptide or natriuretic peptide fragment, and a first specific binding partner labeled with a detectable label, wherein the first specific binding partner is a natriuretic peptide or a natriuretic peptide fragment and further wherein the one or more autoantibodies and the first specific binding partner form a first specific binding partner-autoantibody complex; and
(b) measuring the signal generated by or emitted from the detectable label.

2. The method of claim 1, wherein the natriuretic peptide is a pre-pro peptide precursor of human ANP, a pro peptide of human ANP, a N-terminal pro peptide of ANP, human ANP, a pre-pro peptide precursor of human BNP, a pro peptide of human BNP, a N-terminal pro peptide of BNP, human BNP, human CNP, a pro peptide of human CNP, Dendroaspis natriuretic peptide, a natriuretic peptide fragment, or any combinations thereof.

3. The method of claim 1, wherein the detectable label is an acridinium compound.

4. The method of claim 1, wherein the acridinium compound:
(a) is an acridinium-9-carboxamide having a structure according to formula I: 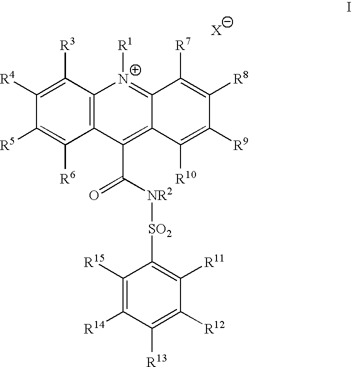
wherein R¹ and R² are each independently selected from the group consisting of: alkyl, alkenyl, alkynyl, aryl or aralkyl, sulfoalkyl carboxyalkyl, oxoalkyl, and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, oxoalkyl and carboxyalkyl; and
optionally, if present, X^{⊖} is an anion; or
(b) is an acridinium-9-carboxylate aryl ester having a structure according to formula II: 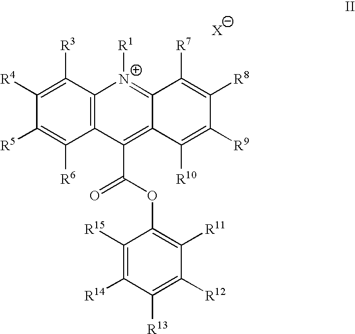
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

5. The method of claim 3, wherein the method further comprises the steps of:
generating in or providing to the mixture a source of hydrogen peroxide before or after the addition of the first specific binding partner in step (a);
adding a basic solution to the mixture to generate a light signal; and
measuring the signal in step (b) by measuring the light generated to detect the one or more autoantibodies.

6. The method of claim 1, wherein the method relates the amount of signal in step (b) to the amount of the one or more autoantibodies in the test sample either by use of a standard curve for the analyte, or by comparison to a reference standard.

7. The method of claim 1, wherein the method is adapted for use in an automated system or semi-automated system.

8. A method for detecting one or more autoantibodies reactive with at least one natriuretic peptide or natriuretic peptide fragment in a test sample, the method comprising the steps of:
(a) preparing a mixture comprising a test sample being assessed for the presence of one or more autoantibodies to at least one natriuretic peptide or natriuretic peptide fragment and a first specific binding partner that is immobilized on a solid phase, wherein the first specific binding partner is a natriuretic peptide or a natriuretic peptide fragment and further wherein the one or more autoantibodies and the first specific binding partner form a solid phase first specific binding partner-autoantibody complex;
(b) adding a second specific binding partner labeled with a detectable label to the mixture to form a first specific binding partner-one or more autoantibodies-second specific binding partner complex, wherein the second specific binding partner is an anti-human antibody; and
(c) measuring the signal generated by or emitted from the detectable label.

9. The method of claim 8, wherein said method further comprises an additional step selected from the group consisting of:
(1) removing any unbound one or more autoantibodies from the solid phase first specific binding partner-autoantibody complex prior to step (b); and
(2) removing any unbound second specific binding partner labeled with a detectable label from the first specific binding partner-one or more autoantibodies-second specific binding partner complex prior to step (c).

10. The method of claim 8, wherein the natriuretic peptide is a pre-pro peptide precursor of human ANP, a pro peptide of human ANP, a N-terminal pro peptide of ANP, human ANP, a pre-pro peptide precursor of human BNP, a pro peptide of human BNP, a N-terminal pro peptide of BNP, human BNP, human CNP, a pro peptide of human CNP, Dendroaspis natriuretic peptide, a natriuretic peptide fragment or any combinations thereof.

11. The method of claim 8, wherein the detectable label is an acridinium compound.

12. The method of claim 11, wherein the acridinium compound:
(a) is an acridinium-9-carboxamide having a structure according to formula I: 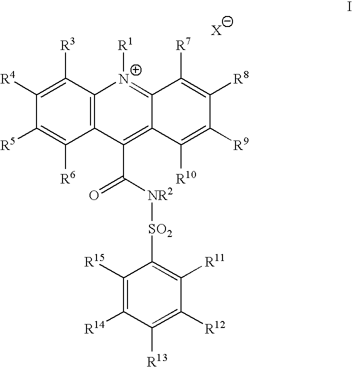
wherein R¹ and R² are each independently selected from the group consisting of: alkyl, alkenyl, alkynyl, aryl or aralkyl, sulfoalkyl carboxyalkyl, oxoalkyl, and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, oxoalkyl and carboxyalkyl; and
optionally, if present, X^{⊖} is an anion; or
(b) is an acridinium-9-carboxylate aryl ester having a structure according to formula II: 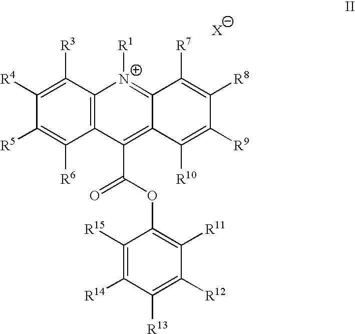
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

13. The method of claim 8, wherein the method further comprises the steps of:
(i) generating in or providing to the mixture a source of hydrogen peroxide before or after the addition of the second specific binding partner containing the detectable label in step (b);
(ii) after step (b) and before step (c), adding a basic solution to the mixture to generate a light signal; and
(iii) measuring the signal in step (c) by measuring the light generated to detect the one or more autoantibodies.

14. The method of claim 8, wherein the method relates the amount of signal in step (c) to the amount of the one or more autoantibodies in the test sample either by use of a standard curve for the analyte, or by comparison to a reference standard.

15. A kit for detecting one or more autoantibodies reactive with at least one natriuretic peptide or a natriuretic peptide fragment in a test sample, the kit comprising:
(a) at least one natriuretic peptide or natriuretic peptide fragment;
(b) at least one detectable label; and
(c) instructions for detecting said one or more autoantibodies.

16. The kit of claim 15, further comprising at least one anti-human antibody.

17. The kit of claim 15, wherein the natriuretic peptide is a pre-pro peptide precursor of human ANP, a pro peptide of human ANP, a N-terminal pro peptide of ANP, human ANP, a pre-pro peptide precursor of human BNP, a pro peptide of human BNP, a N-terminal pro peptide of BNP, human BNP, human CNP, a pro peptide of human CNP, Dendroaspis natriuretic peptide, a natriuretic peptide fragment, or any combinations thereof.

18. The kit of claim 15, wherein the detectable label is an acridinium compound.

19. The kit of claim 18, wherein the acridinium compound is:
(a) an acridinium-9-carboxamide having a structure according to formula I: 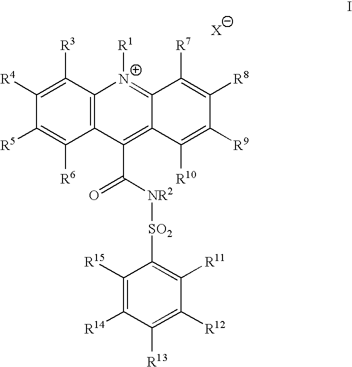
wherein R¹ and R² are each independently selected from the group consisting of: alkyl, alkenyl, alkynyl, aryl or aralkyl, sulfoalkyl oxoalkyl and carboxyalkyl, and
wherein R³ through R¹⁵ are each independently selected from the group consisting of: hydrogen; alkyl, alkenyl, alkynyl, aryl or aralkyl, amino, amido, acyl, alkoxyl, hydroxyl, carboxyl, halide, nitro, cyano, sulfo, sulfoalkyl, oxoalkyl and carboxyalkyl; and
optionally, if present, X^{⊖} is an anion; or
(b) is an acridinium-9-carboxylate aryl ester having a structure according to formula II: 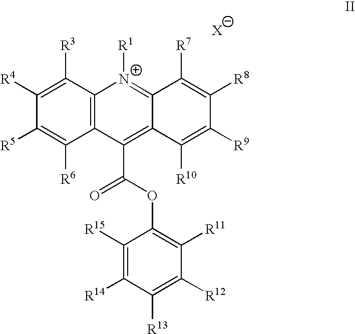
wherein R¹ is an alkyl, alkenyl, alkynyl, aryl or aralkyl, sulfoalkyl, carboxyalkyl and oxoalkyl; and
wherein R³ through R¹⁵ are each independently selected from the group consisting of: hydrogen, alkyl, alkenyl, alkynyl, aryl or aralkyl, amino, amido, acyl, alkoxyl, hydroxyl, carboxyl, halogen, halide, nitro, cyano, sulfo, sulfoalkyl, carboxyalkyl and oxoalkyl; and
optionally, if present, X^{⊖} is an anion.

20. The kit of claim 18, wherein the kit further comprises:
(d) at least one basic solution; and
(e) a source of hydrogen peroxide, wherein said source contains a predetermined amount of hydrogen peroxide.

21. The kit of claim 20, wherein the kit further comprises at least one anti-human antibody.

22. An isolated human natriuretic peptide autoantibody, wherein the autoantibody is obtained by a process comprising the steps of:
(a) preparing a mixture comprising a human natriuretic peptide autoantibody; and
(b) isolating the human natriuretic peptide autoantibody from the mixture.

23. The antibody of claim 22, wherein the antibody is an IgG antibody.

24. The antibody of claim 22, wherein the antibody is an isolated human autoantibody selected from the group consisting of: a pre-pro peptide precursor of human BNP autoantibody, a pro peptide of human BNP autoantibody, a N-terminal pro peptide of BNP autoantibody and a human BNP autoantibody.

25. The antibody of claim 22, wherein the antibody is an isolated pro peptide human BNP autoantibody.

26. A method of screening for at least one agent useful in inhibiting the binding of at least one human natriuretic peptide or natriuretic peptide fragment to at least one human natriuretic peptide autoantibody, the method comprising the steps of:
(a) preparing a mixture comprising an isolated human natriuretic peptide autoantibody;
(b) adding to the mixture at least one human natriuretic peptide or natriuretic peptide fragment and at least one agent to be tested; and
(c) determining whether the agent inhibits the binding of the at least one human natriuretic peptide or natriuretic peptide fragment to the human natriuretic peptide autoantibody.

27. A method of determining the reliability of a human natriuretic peptide assay result, the method comprising the steps of:
(a) assaying a test sample for one or more autoantibodies reactive with a human natriuretic peptide; and
(b) determining the reliability of a human natriuretic peptide assay result, wherein the presence of an elevated level in the test sample of one or more autoantibodies reactive with a human natriuretic peptide indicates that the human natriuretic peptide result is not reliable.

28. A method of assessing whether a subject has or is at risk of developing cardiovascular disease, the method comprising the steps of:
(a) determining the concentration or amount in a test sample from a subject of one or more autoantibodies reactive with human natriuretic peptide; and
(b) comparing the concentration or amount of the one or more autoantibodies reactive with human natriuretic peptide determined in step (a) with a predetermined level, wherein if the concentration or amount of the one or more autoantibodies reactive with human natriuretic peptide determined in step (a) is favorable with respect to a predetermined level, then the subject is determined not to have or be at risk for a cardiovascular disease; and further wherein if the concentration or amount of the one or more autoantibodies reactive with human natriuretic peptide determined in step (a) is unfavorable with respect to the predetermined level then the subject is determined to have or be at risk for a cardiovascular disease.

29. The method of claim 28, wherein the human natriuretic peptide is a pre-pro peptide precursor of human ANP, a pro peptide of human ANP, a N-terminal pro peptide of ANP, human ANP, a pre-pro peptide precursor of human BNP, a pro peptide of human BNP, a N-terminal pro peptide of BNP, human BNP, human CNP, a pro peptide of human CNP or any combinations thereof.

30. A method of monitoring the progression of disease in a subject, the method comprising the steps of:
(a) determining the concentration or amount in a first test sample from a subject of one or more autoantibodies reactive with human natriuretic peptide;
(b) determining the concentration or amount in a second or subsequent test sample from the subject of one or more autoantibodies reactive with human natriuretic peptide; and
(c) comparing the concentration or amount of one or more autoantibodies reactive with human natriuretic peptide determined in step (b) with the concentration or amount of one or more autoantibodies reactive with human natriuretic peptide determined in step (a),
wherein if the concentration or amount determined in step (b) is unchanged or is unfavorable when compared to the concentration or amount of one or more autoantibodies reactive with human natriuretic peptide determined in step (a), then the disease in the subject is determined to have continued, progressed or worsened,
further wherein, if the concentration or amount of one or more autoantibodies reactive with human natriuretic peptide determined in step (b) is favorable when compared to the concentration or amount of one or more autoantibodies reactive with human natriuretic peptide determined in step (a), then the disease in the subject is determined to have discontinued, regressed or improved.

31. The method of claim 30, wherein said method further comprises comparing the concentration or amount of one or more autoantibodies reactive with human natriuretic peptide determined in step (b) or step (d) with a predetermined level.

32. The method of claim 30, wherein said method further comprises treating the subject with one or more pharmaceutical compositions for a period of time if the comparison shows that the concentration or amount of one or more autoantibodies reactive with human natriuretic peptide determined in step (b) or step (d) is unfavorably altered with respect to the predetermined level.

33. A method of monitoring the treatment of disease in a subject, the method comprising the steps of:
(a) determining the concentration or amount in a first test sample from a subject of one or more autoantibodies reactive with human natriuretic peptide;
(b) treating the subject with one or more pharmaceutical compositions for a period of time;
(c) determining the concentration or amount in a second or subsequent test sample obtained from the subject following treatment in step (b) of one or more autoantibodies reactive with human natriuretic peptide; and
(d) comparing the concentration or amount of one or more autoantibodies reactive with human natriuretic peptide determined in step (c) with the concentration or amount of one or more autoantibodies reactive with human natriuretic peptide determined in step (a),
wherein if the concentration or amount determined in step (c) is unchanged or is unfavorable when compared to the concentration or amount of one or more autoantibodies reactive with human natriuretic peptide determined in step (a), then the subject should be treated with a higher concentration of the one or more pharmaceutical compositions administered to the subject in step (b) or the subject should be treated with one or more pharmaceutical compositions that are different then the one or more pharmaceutical compositions administered to the subject in step (b).
further wherein, if the concentration or amount of one or more autoantibodies reactive with human natriuretic peptide determined in step (c) is favorable when compared to the concentration or amount of one or more autoantibodies reactive with human natriuretic peptide determined in step (a), then the subject should continue to be administered the one or pharmaceutical compositions of step (b).

34. The method of claim 33, wherein said method further comprises comparing the concentration or amount of one or more autoantibodies reactive with human natriuretic peptide determined in step (a) or step (c) with a predetermined level.
